SEQUENCE LISTING (44 sequences)

SEQ ID NO: 1 - protein, 23 aa, Artificial Sequence
  DSAR2 zinc finger module
YSCGICGKSFSDSSAKRRHCILH

SEQ ID NO: 2 - protein, 23 aa, Artificial Sequence
  DSCR zinc finger module
YTCSDCGKAFRDKSCLNRHRRTH

SEQ ID NO: 3 - protein, 24 aa, Artificial Sequence
  DSNR zinc finger module
YRCKYCDRSFSDSSNLQRHVRNIH

SEQ ID NO: 4 - protein, 24 aa, Artificial Sequence
  ISNR zinc finger module
YRCKYCDRSFSISSNLQRHVRNIH

SEQ ID NO: 5 - protein, 23 aa, Artificial Sequence
  KSNR zinc finger module
YGCHLCGKAFSKSSNLRRHEMIH

SEQ ID NO: 6 - protein, 23 aa, Artificial Sequence
  QNTQ zinc finger module
YTCSYCGKSFTQSNTLKQHTRIH

SEQ ID NO: 7 - protein, 23 aa, Artificial Sequence
  QSHR2 zinc finger module
YKCGQCGKFYSQVSHLTRHQKIH

SEQ ID NO: 8 - protein, 23 aa, Artificial Sequence
  QSHV zinc finger module
YECDHCGKSFSQSSHLNVHKRTH

SEQ ID NO: 9 - protein, 23 aa, Artificial Sequence
  QSNR1 zinc finger module
FECKDCGKAFIQKSNLIRHQRTH

SEQ ID NO: 10 - protein, 23 aa, Artificial Sequence
  QSSR1 zinc finger module
YKCPDCGKSFSQSSSLIRHQRTH

SEQ ID NO: 11 - protein, 25 aa, Artificial Sequence
  RDER2 zinc finger module
YHCDWDGCGWKFARSDELTRHYRKH

SEQ ID NO: 12 - protein, 23 aa, Artificial Sequence
  RDHT zinc finger module
FQCKTCQRKFSRSDHLKTHTRTH

SEQ ID NO: 13 - protein, 23 aa, Artificial Sequence
  RSHR zinc finger module
YKCMECGKAFNRRSHLTRHQRIH

SEQ ID NO: 14 - protein, 23 aa, Artificial Sequence
  VDYK zinc finger module
FHCGYCEKSFSVKDYLTKHIRTH

SEQ ID NO: 15 - protein, 23 aa, Artificial Sequence
  VSNV zinc finger module
YECDHCGKAFSVSSNLNVHRRIH

SEQ ID NO: 16 - protein, 23 aa, Artificial Sequence
  VSTR zinc finger module
YECNYCGKTFSVSSTLIRHQRIH

SEQ ID NO: 17 - protein, 23 aa, Artificial Sequence
  WSNR zinc finger module
YRCEECGKAFRWPSNLTRHKRIH

SEQ ID NO: 18 - DNA, 20 bp, Artificial Sequence
  F2 primer
ccacatctcgttctcggttt

SEQ ID NO: 19 - DNA, 21 bp, Artificial Sequence
  R2 primer
gcacctgctttacaggtttct

SEQ ID NO: 20 - DNA, 20 bp, Artificial Sequence
  F5 primer
atggattatcaagtgtcaag

SEQ ID NO: 21 - DNA, 20 bp, Artificial Sequence
  R5 primer
tcacaagcccacagatattt

SEQ ID NO: 22 - DNA, 19 bp, Artificial Sequence
  R-S162 primer
gtatggaaaatgagagctg

SEQ ID NO: 23 - DNA, 20 bp, Artificial Sequence
  F-K33 primer
agcatggttcagaaggccac

SEQ ID NO: 24 - DNA, 22 bp, Artificial Sequence
  R-K33 primer
tggctgagtagtattccatggt

SEQ ID NO: 25 - DNA, 21 bp, Artificial Sequence
  F-K230 primer
gggagctgaaataccttcctt

SEQ ID NO: 26 - DNA, 20 bp, Artificial Sequence
  R-K230 primer
atgtggcatcacacatggag

SEQ ID NO: 27 - DNA, 21 bp, Artificial Sequence
  F-K243 primer
gccgggtttgtacaaggtaga

SEQ ID NO: 28 - DNA, 20 bp, Artificial Sequence
  R-K243 primer
ccctgtgttcccttctaagc

SEQ ID NO: 29 - DNA, 20 bp, Artificial Sequence
  F-K276 primer
atccctgcctcacagctcat

SEQ ID NO: 30 - DNA, 20 bp, Artificial Sequence
  R-K276 primer
ttagttcctggtttggtgcc

SEQ ID NO: 31 - DNA, 20 bp, Artificial Sequence
  F-K781 primer
tgcaggtacatgccgaactg

SEQ ID NO: 32 - DNA, 20 bp, Artificial Sequence
  R-K781 primer
cctaccatcccctttctcag

SEQ ID NO: 33 - DNA, 22 bp, Artificial Sequence
  F-K835 primer
cccactgatgctctgatagttt

SEQ ID NO: 34 - DNA, 20 bp, Artificial Sequence
  R-K835 primer
tgggagatgaaaggaccttg

SEQ ID NO: 35 - DNA, 21 bp, Artificial Sequence
  F-M15 primer
cgagaaggaaacctagcaagg

SEQ ID NO: 36 - DNA, 20 bp, Artificial Sequence
  R-M15 primer
caattactccccaggtgtcc

SEQ ID NO: 37 - DNA, 22 bp, Artificial Sequence
  Z30F primer
tgcacagggtggaacaagatgg

SEQ ID NO: 38 - DNA, 26 bp, Artificial Sequence
  S162R primer
gagcccagaaggggacagtaagaagg

SEQ ID NO: 39 - DNA, 28 bp, Artificial Sequence
  S162 5F primer
ctgatttgagtgaattctcacgtgacag

SEQ ID NO: 40 - DNA, 28 bp, Artificial Sequence
  S162 5R primer
atcagctgtcacgtgagaattcactcaa

SEQ ID NO: 41 - DNA, 27 bp, Artificial Sequence
  F-891 primer
ccttttgagtgaattctcacgtgacag

SEQ ID NO: 42 - DNA, 27 bp, Artificial Sequence
  OR-891 primer
aaggctgtcacgtgagaattcactcaa

SEQ ID NO: 43 - DNA, 28 bp, Artificial Sequence
  2-F-162 primer
ttaatttgagtgaattctcacgtgacag

SEQ ID NO: 44 - DNA, 28 bp, Artificial Sequence
  5-OR-162 primer
atcagctgtcacgtgagaattcactcaa

(19) United States
(12) Patent Application Publication
(10) Pub. No.: US 2012/0149115 A1
(43) Pub. Date: Jun. 14, 2012

(54) TARGETED GENOMIC REARRANGEMENTS USING SITE-SPECIFIC NUCLEASES

(75) Inventors: Jin Soo Kim, Seoul (KR); Hyung Joo Lee, Gyeonggi-do (KR); Eun Ji Kim, Seoul (KR)

(73) Assignees: SNU R&DB FOUNDATION, Seoul (KR); TOOLGEN INCORPORATION, Seoul (KR)

(21) Appl. No.: 13/377,387

(22) PCT Filed: Jun. 11, 2010
(86) PCT No.: PCT/KR2010/003786

§ 371 Date: Feb. 22, 2012

(30) Foreign Application Priority Data
Jun. 11, 2009 (KR) 10-2009-0051896

(51) Int. Cl. C12N 15/01; C12N 5/071; C12N 5/04; C12N 1/16; C12N 1/14; C12N 1/10; C12N 9/16; C12N 1/20

(52) U.S. Cl. 435/441; 435/196; 435/369; 435/252.8; 435/255.1; 435/254.1; 435/258.1; 435/325; 435/348; 435/358; 435/367; 435/365; 435/366

(57) ABSTRACT

The present invention relates to a method for genomic DNA rearrangements, and more particularly, to a method for deletion, duplication, inversion, replacement, or rearrangement of genomic DNA using pairs of site-specific nucleases targeting two or more sites in the genome, a cell in which genomic DNA is deleted, duplicated, inverted, replaced, or rearranged by the same method, and a method for expressing the site-specific nucleases in cells. Further, the present invention relates to a method for inserting synthetic DNA molecules into the genome using site-specific nucleases targeting a pre-determined site in the genome, a cell in which DNA insertion occurs by the same method, and a method for expressing the site-specific nucleases in cells.

### TECHNICAL FIELD

The present invention relates to a method for genomic DNA rearrangements, and more particularly, to a method for deletion, duplication, inversion, replacement, or re-arrangement of genomic DNA using site-specific nucleases targeting two or more sites in the genome, a cell in which genomic DNA is deleted, duplicated, inverted, replaced, or rearranged by the same method, and a method for expressing the site-specific nucleases in cells. Further, the present invention relates to a method for inserting synthetic DNA molecules into the genome using site-specific nucleases targeting a pre-determined site in the genome, a cell in which DNA insertion occurs by the same method, and a method for expressing the site-specific nucleases in cells.

### BACKGROUND ART

Recombinant DNA technology widely used in modern genetic engineering has played a pivotal role in the advancement of the life sciences and has paved the way for biotechnology. Restriction endonucleases are key components in recombinant DNA technology, which allow cutting and pasting DNA segments in vitro. But these enzymes cleave to DNA too frequently and therefore cannot be used for manipulation of genomic scripts in cells. Thus, recombinant DNA technology has been limited to the use of restriction enzymes in vitro but not in vivo. Thanks to the next generation DNA sequencing technologies, genomes of many organisms have been sequenced in an accelerated phase, and rapid progress is demanded for genome engineering in the post-genomic era. In-cell recombinant DNA technology, which is defined herein as a novel genome engineering approach that allows i) targeted deletions of genomic DNA segments in higher eukaryotic cells and organisms, ii) targeted insertions of synthetic oligodeoxynucleotides (ODN) cassettes into pre-determined sites in the genome, and iii) replacements of genomic DNA segments with synthetic DNA molecules, could add new dimensions to biology, medicine and biotechnology.

Current methods of genome engineering fall short of being the versatile tools as in-cell recombinant DNA technology. For example, the method of random integration of foreign DNA segments into the genome, which is most commonly used for genome engineering, suffers from possible disruption of endogenous genes or activation of unwanted genes due to the accidental insertion of promoter or enhancer elements associated with the exogenous segments. Gene targeting via homologous recombination (HR) allows precise manipulation of genomic scripts, but is of limited use for most higher eukaryotic cells and organisms due to its poor efficiency. Recombinases such as Cre can be used as tools for genome engineering, but these enzymes require pre-insertion of their own recognition elements into the genome, which are left behind in the genome after enzymatic treatments. Transposases also leave footprints in the genome and do not allow targeted manipulation of genomic scripts.

The ability to generate targeted deletions of genomic DNA greater than 10 kbp in length could expand genetic and genomic studies into new dimensions by allowing the selective removal of gene clusters, intergenic regions, exons and introns from a genome and may have broad applications in research, biotechnology and gene therapy, but it has been difficult, if not impossible, to achieve this aim in higher eukaryotic cells and organisms. The Cre/loxP method (Ramirez-Solis et al. 1995) or BAC-based gene targeting (Valenzuela et al. 2003) in murine embryonic stem cells has been used for targeted deletion of large genomic DNA segments. However, these approaches are practically limited to murine embryonic stem cells, which are more amenable to genetic manipulation via homologous recombination than are other cells. Furthermore, the Cre/loxP method requires two rounds of loxP insertion into the genome via homologous recombination (HR), isolation of cells in which two target sites are inserted in the same chromosome but not in different homologous chromosomes, and subsequent treatment with Cre recombinases to delete the intervening DNA segment, a process that still leaves a single loxP site behind in the genome after targeted deletion. BAC-based gene targeting also has limitations associated with the preparation of BAC vectors and the screening of recombinant clones due to the huge size of these vectors. In addition, false positive clones are often isolated, which result from the breakage and partial integration of BAC vectors (Gomez-Rodriguez et al. 2008). Thus, these approaches are highly laborious and time-consuming even in murine embryonic stem cells, and, to our knowledge, have never been used to delete pre-determined genomic DNA segments in other higher mammalian or plant cells.

Site-specific nucleases refer to all of the enzymes capable of specifically recognizing and cleaving DNA sequences, and Zinc finger nucleases, one of the site-specific nucleases, (hereinbelow, referred to as “ZFN”) are promising new tools for genome engineering. ZFNs are artificial enzymes that consist of a DNA-binding zinc finger domain and DNA cleavage domain derived from a Fokl nuclease. Unlike conventional recombinases, ZFNs are reprogrammable and tailor-made nucleases could be produced easily to target any pre-determined endogenous sites in the genome. ZFNs recognize a target sequence to induce site-specific DNA double-strand breaks (hereinbelow, referred to as “DSB”) in cells, which are repaired by two endogenous mechanisms known as homologous recombination (HR) and non-homologous end joining (NHEJ), giving rise to targeted mutagenesis.

ZFNs have been shown to inactivate endogenous genes of interest or introduce specific mutations in mammalian cells, plant, zebrafish, and fruitfly. However, all these previous efforts demonstrated the utilities of ZFNs to induce site-specific, local mutations at pre-determined sites in the genome but did not involve targeted deletions of genomic DNA segments in cells, site-specific insertions of synthetic oligonucleotide (dsODN) cassettes into the genome, nor targeted replacements of endogenous DNA segments with synthetic DNA elements in cells. Thus, ZFNs hold promise in genome engineering but, thus far, still come short of versatile tools as in-cell recombinant DNA technology.

### DISCLOSURE OF INVENTION

### Technical Problem

Therefore, the present inventors have made many efforts to find ZFNs capable of inducing genome deletions, insertions and replacements. We found that ZFNs designed to target two different sites in a human chromosome could introduce two DSBs in the chromosome and give rise to targeted deletions of the genomic DNA segments in the range of several hundred base pair to 15 Mbp, thereby completing the present invention.

### Solution to Problem

It is an object of the present invention to provide a method for deletion, duplication, inversion, replacement, or rearrangement of genomic DNA, comprising the steps of cleaving two or more pre-determined sites in a genome using site-specific nucleases.

It is another object of the present invention to provide a cell in which a genomic DNA segment is deleted, duplicated, inverted, replaced, or rearranged by the same method.

It is still another object of the present invention to provide a method for inserting synthetic DNA molecules, comprising the step of cleaving a pre-determined site in a genome using site-specific nucleases.

It is still another object of the present invention to provide a cell in which synthetic DNA molecules are inserted into a pre-determined genomic site by the same method.

It is still another object of the present invention to provide a method for expressing the zinc finger nucleases in cells, comprising the steps of (a) determining a specific base sequence to be cleaved; (b) selecting zinc finger modules to recognize the base sequence; (c) preparing zinc finger nucleases including the zinc finger modules of step (b); and (d) introducing the prepared zinc finger nucleases into cells.

### Advantageous Effects of Invention

Site-specific nucleases, specifically zinc finger nucleases of the present invention capable of deletion, duplication, inversion, replacement, insertion, or rearrangement of large genomic DNA segments can be used to remove gene clusters from the genome of interest via targeted genome deletions, or can be applied to stem cell research and gene therapy. In addition, they can be employed to create useful organisms by increasing the gene dosage of plants or animals via duplication, or used as a tools for the treatment of cancer cells and cells of patients with inherited diseases via inversion, or employed for the breed improvement of crops, fish, and livestock animals via replacement and insertion, or ultimately, used to induce targeted mutations of the desired genes via site-specific nucleases, specifically zinc finger nuclease-induced genome surgery.

### BRIEF DESCRIPTION OF DRAWINGS

FIG. 1 is a schematic diagram showing the ZFN-induced genome deletions at the CCR2 and CCR5 loci, in which the zigzag lines indicate ZFN target sites, and F2 and R5 (arrows) are PCR primers used for the detection of genome deletion events (DSB: double strand break);

FIG. 2 shows PCR products corresponding to the genomic DNA deletions in cells treated with ZFNs, in which p3 is the empty plasmid used as a negative control;

FIG. 3 shows DNA sequences of PCR products, in which PCR products were cloned and sequenced, ZFN target sites are shown in bold letters, microhomologies are underlined and inserted bases are shown in italics, dashes (-) indicate deleted bases, non-conserved bases at the CCR2 and CCR5 loci are shown in small letters, the number of occurrences is shown in parentheses, and WT indicates wild-type DNA sequence;

FIG. 4 is a schematic diagram showing two different deletion events by ZFNs within the CCR5 locus, in which F5 and R5 (arrows) are primers used for the amplification of the CCR5 coding base sequence;

FIG. 5 is the result of PCR analysis showing two different deletion events within the CCR5 locus, in which as expected in FIG. 4, approximate sizes of PCR products corresponding to deletion events ((1)199 bp and (2)331 bp) and to the intact wild-type sequence (1,060 bp) are shown, and p3 is the empty plasmid used as a negative control;

FIG. 6 shows DNA sequences of breakpoint junctions of two ZFN-induced genome deletions within the CCR5 locus, in which ZFN target sites are shown in bold letters, microhomologies are underlined and inserted bases are shown in italics, dashes (-) indicate deleted bases, non-conserved bases at the CCR2 and CCR5 loci are shown in small letters, the number of occurrences is shown in parentheses, and WT indicates wild-type DNA sequence;

FIG. 7 shows large nested genomic deletions of target sites on a chromosomal ideogram, in which arrows indicate the locations of ZFN target sites on the amplified view of the relevant chromosome 3 region;

FIG. 8 is the result of PCR analysis showing large nested deletions, in which S162 and each of the seven new ZFNs were co-expressed in HEK 293 cells, and the sequences of primers used in the PCR analysis are summarized in Table 2;

FIG. 9 shows DNA sequences of breakpoint junctions of large deletions, in which ZFN target sites are shown in bold letters, microhomologies are underlined and inserted bases are shown in italics, dashes (-) indicate deleted bases, the number of occurrences is shown in parentheses, and WT indicates wild-type DNA sequence;

FIG. 10 shows DNA sequences of breakpoint junctions of large deletions, in which ZFN target sites are shown in bold letters, microhomologies are underlined and inserted bases are shown in italics, dashes (-) indicate deleted bases, the number of occurrences is shown in parentheses, and WT indicates wild-type DNA sequence;

FIG. 11 is a schematic diagram showing the ZFN-induced duplication, in which F5 and R2 (arrows) are PCR primers used for the detection of genome duplication events;

FIG. 12 is the result of PCR analysis showing the ZFN-induced duplication, in which p3 is the empty plasmid used as a negative control;

FIG. 13 shows DNA sequences of breakpoint junctions of the ZFN-induced duplication, in which each base sequence of CCR5 and CCR2 are represented by black and gray colors, the duplicated base sequences are direct connection of the 5′ parts of the CCR5 coding region and 3′ parts of the CCR2 coding region, ZFN target sites are shown in bold letters, microhomologies are underlined and inserted bases are shown in italics, dashes (-) indicate deleted bases, non-conserved bases at the CCR2 and CCR5 loci are shown in small letters, and the number of occurrences is shown in parentheses;

FIG. 14 shows DNA sequences of breakpoint junctions of the duplication induced by new ZFN combinations, in which ZFN target sites are shown in bold letters, microhomologies are underlined and inserted bases are shown in italics, dashes (-) indicate deleted bases, and the number of occurrences is shown in parenthesese;

FIG. 15 is a schematic diagram showing the ZFN-induced inversion, in which the PCR primers F_{A} and F_{B} were used for the detection of Breakpoint junction 1 and the PCR primers R_{A} and R_{B} were used for the detection of Breakpoint junction 2, “Cleaved” indicates the ZFN-induced genome cleavage, “Flipped” indicates 180° rotation of the cleaved DNA, and “Inverted” indicates joining of the cleaved sites by non-homologous end-joining (NHEJ);

FIG. 16 is the result of PCR analysis showing the ZFN-induced inversion, in which S162 and each of the six new ZFNs were co-expressed in HEK 293 cells, and the sequences of primers used in the PCR analysis are summarized in Table 2;

FIG. 17 shows DNA sequences of breakpoint junctions of the inversion, in which ZFN target sites are shown in bold letters, microhomologies are underlined and inserted bases are shown in italics, dashes (-) indicate deleted bases, the number of occurrences is shown in parentheses, and WT indicates wild-type DNA sequence;

FIG. 18 shows DNA sequences of breakpoint junction 1 of the inversion, in which ZFN target sites are shown in bold letters, microhomologies are underlined and inserted bases are shown in italics, dashes (-) indicate deleted bases, and the number of occurrences is shown in parentheses;

FIG. 19 shows DNA sequences of breakpoint junction 2 of the inversion, in which ZFN target sites are shown in bold letters, microhomologies are underlined and inserted bases are shown in italics, dashes (-) indicate deleted bases, and the number of occurrences is shown in parentheses;

FIG. 20 is the analysis result of clonal populations of cells, and shows DNA sequences of ZFN target sites at CCR2 and CCR5 loci of cloned cells whose genomic segments were deleted, in which ZFN target sites are shown in bold letters, microhomologies are underlined and inserted bases are shown in italics, dashes (-) indicate deleted bases, and WT indicates wild-type DNA sequence;

FIG. 21 is the result of Southern blot analysis of clonal cells, in which a 9.7-kb band corresponding to the genomic deletion was detected using DNAs around the CCR2 locus as a probe, X indicates the XbaI restriction site, S162 indicates the ZFN target site, the white arrow indicates the CCR2 coding region, the gray arrow indicates the CCR5 coding region, and WT indicates wild-type HEK293 cells;

FIG. 22 is a schematic diagram showing the ZFN-induced insertion and replacement, in which the zigzag lines indicate ZFN target sites, and F and R (arrows) are PCR primers used for the detection of genome insertion and deletion events, OF and OR indicate oligonucleotides, and dsODN cassette was prepared by the annealing of OF and OR;

FIG. 23 is the result of PCR analysis of genomic DNA of ZFN and synthetic DNA-treated cells, in which p3 is the empty plasmid used as a negative control, OR-30 and OR-891 indicate one strand of ODN constituting the dsODN cassette, and F-30 and F-891 are primers used for the detection of synthetic DNA insertion;

FIG. 24 shows DNA sequences of PCR products obtained from Z30 and dsODN cassette-treated cells, in which PCR products were cloned and sequenced, ZFN target sites are shown in bold letters, dashes (-) indicate deleted bases, the number of occurrences is shown in parentheses, and WT indicates wild-type DNA sequence;

FIG. 25 shows DNA sequences of PCR products obtained from Z891 and dsODN cassette-treated cells, in which PCR products were cloned and sequenced, ZFN target sites are shown in bold letters, dashes (-) indicate deleted bases, the number of occurrences is shown in parentheses, and WT indicates wild-type DNA sequence;

FIGS. 26 to 28 show ZFN-induced dsODN (double strand oligonucleotide) insertion at the CCR5 locus. Fluorescent PCR analysis of (FIG. 26) genomic DNA isolated from control cells, (FIG. 27) genomic DNA isolated from cells treated with ZFN and (FIG. 28) genomic DNA isolated from cells treated with ZFN and dsODNs. Single asterisks (*) represent DNA peaks corresponding to ZFN-induced deletions. Double asterisks (**) represent ZFN-induced specific 5-bp insertion, and triple asterisks (***) represent ZFN-induced genomic insertion of dsODN. Note that insertion peaks are not present in the control (FIG. 26 and FIG. 27). The numbers indicate peak areas that are pro-portional to amounts of DNA; and

FIG. 29 shows targeted replacement of genomic DNA segments. After performing PCR capable of identifying the deletion of 15-kbp DNA between CCR2 and CCR5 from cells treated with ZFN, the DNA was cleaved by EcoRI. Each of the lanes shows the result of cleaving cell genomes wherein p3 used as a negative control is treated; ZFN-treated cells; and cells treated with both ZFN and dsODN cassette using EcoRI. The arrow indicates the DNA segment cleaved by dsODN cassette-derived EcoRI.

### BEST MODE FOR CARRYING OUT THE INVENTION

In one aspect, to achieve the above objects, the present invention relates to a method for deletion, duplication, inversion, replacement, or rearrangement of genomic DNA, comprising the step of cleaving two or more pre-determined sites in a genome using site-specific nucleases.

The term “site-specific nuclease” of the present invention refers to a nuclease capable of recognizing and cleaving target sites of DNA in the genome and, for the purpose of the present invention, can include a nuclease wherein a domain recognizing target sites in the genome and a cleavage domain are fused. As examples, a modified meganuclease, a fusion protein wherein a TAL effector (transcription activator-like effector) domain derived from a phytopathogenic gene (which is a domain recognizing target sites in the genome) and a cleavage domain are fused, and a zinc finger nuclease can be included without limitation, but the most preferable one is a zinc finger nuclease.

The term “modified meganuclease” of the present invention refers to an enzyme being modified to enable the restriction enzyme “meganuclease” capable of recognizing over 10 bp of DNA in the genome to have a new DNA cleavage specificity using a pre-existing molecular biological method.

As used herein, the term “zinc finger nuclease” refers to a fusion protein comprising a zinc finger domain and a nucleotide cleavage domain, and may include all known or commercial zinc finger nucleases. In addition, the zinc finger nuclease may be, but is not limited to, any one zinc finger nuclease that is described in Table 3 and used in one preferred embodiment. In the present invention, the terms “zinc finger nuclease” and “ZFN” are interchangeable.

Zinc finger nucleases may function as dimmers, for example homodimers or heterodimers, to introduce DNA double strand breaks, thereby achieving the desired object of the present invention.

In general, because zinc finger nucleases (ZFNs) function as dimers, two ZFN monomers need to be prepared to target a single DNA site. Each of two monomeric ZFNs recognizes one of two half-sites in different DNA strands, which are separated from each other by a 5- or 6-bp spacer. In addition, since a single zinc finger module recognizes and binds to 3 bp of subsite, the zinc finger domain consisting of 2 to 4 zinc finger modules recognizes 6 to 12-bp DNA binding sites.

As used herein, the term “zinc finger domain” refers to a protein that binds to a nucleotide in a sequence-specific manner through one or more zinc finger modules. The zinc finger domain includes at least two zinc finger modules. The zinc finger domain is often abbreviated as zinc finger protein or ZFP.

As used herein, the term “zinc finger module” refers to an amino acid sequence within the binding domain whose structure is stabilized through coordination of a zinc ion. The zinc finger modules of the present invention have the sequences being identical to those of the naturally-occurring, wild-type zinc finger modules or the sequences that are modified by substitution of other amino acids for any amino acids in the wild-type sequence. The wild-type zinc finger module may be derived from any eukaryotic cells, for example, fungal cells (e.g., yeast), plant or animal cells, (e.g., mammalian cells such as human or mouse). The zinc finger module to be used in the present invention may include any known modules and commercial modules, but is not limited thereto. Preferably, the zinc finger modules of the present invention may be two or more zinc finger modules, more preferably two to four zinc finger modules, much more preferably three zinc finger modules, and most preferably selected from the modules described in the following Table 1.

TABLE 1
Table 1. Zinc finger modules

| | | | | SEQ |
|---|---|---|---|---|
| ZF | ZF | | Target | ID |
| No.¹ | name | Amino acid sequence | subsite | NO. |
| N.A. | DSAR2 | YSCGICGKSFSDSSAKRRHCILH | GTC | 1 |
| ZF108 | DSCR | YTCSDCGKAFRDKSCLNRHRRTH | GCC | 2 |
| N.A. | DSNR | YRCKYCDRSFSDSSNLQRHVRN | GAC | 3 |
| | | IH | | |
| ZF112 | ISNR | YRCKYCDRSFSISSNLQRHVRN | GAT | 4 |
| | | IH | | |
| ZF113 | KSNR | YGCHLCGKAFSKSSNLRRHEMIH | GAG | 5 |
| N.A. | QNTQ | YTCSYCGKSFTQSNTLKQHTRIH | ATA | 6 |
| ZF117 | QSHR2 | YKCGQCGKFYSQVSHLTRHQKIH | GGA | 7 |
| ZF120 | QSHV | YECDHCGKSFSQSSHLNVHKRTH | YGA | 8 |
| ZF123 | QSNR1 | FECKDCGKAFIQKSNLIRHQRTH | GAA | 9 |
| ZF126 | QSSR1 | YKCPDCGKSFSQSSSLIRHQRTH | GYA | 10 |
| N.A. | RDER2 | YHCDWDGCGWKFARSDELTRHYR | GYG | 11 |
| | | KH | | |
| ZF132 | RDHT | FQCKTCQRKFSRSDHLKTHTRTH | YGG | 12 |
| ZF134 | RSHR | YKCMECGKAFNRRSHLTRHQRIH | GGG | 13 |
| N.A. | VDYK | FHCGYCEKSFSVKDYLTKHIRTH | TAT | 14 |
| ZF137 | VSNV | YECDHCGKAFSVSSNLNVHRRIH | AAT | 15 |
| ZF139 | VSTR | YECNYCGKTFSVSSTLIRHQRIH | GCT | 16 |
| ZF140 | WSNR | YRCEECGKAFRWPSNLTRHKRIH | GGT | 17 |

¹ZF No. is based on the numbering scheme of the Zinc Finger Consortium Modular Assembly Kit 1.0 available from Addgene.

The zinc finger domains of zinc finger nucleases consist of 2 or more tandemly arrayed zinc finger modules, each of which recognizes 3 bp sub-sites. Since each module independently recognizes DNA sequences, the zinc finger domains consisting of 2 to 4 modules are able to bind to a 6- or 12-bp sequence. Zinc finger nucleases function as dimmers and, therefore, a pair of zinc finger nuclease consisting of 2 to 4 zinc finger modules specifically recognizes 12- to 24-bp sequence. In a specific example, the zinc finger nucleases of the present invention have the zinc finger domains consisting of 2 or more, preferably 2 to 4, and more preferably 3 zinc finger modules.

As used herein, the term “cleavage” refers to the breakage of the covalent backbone of a nucleotide molecule, and the term “cleavage domain” refers to a polypeptide sequences which possesses catalytic activity for nucleotide cleavage.

The cleavage domain can be obtained from any endo- or exonuclease. Exemplary endonucleases from which a cleavage domain can be derived include, but are not limited to, restriction endonucleases. These enzymes can be used as a source of cleavage domains. In addition, the cleavage domain is able to cleave single-stranded nucleotide sequences, in which double-stranded cleavage can occur depending on the source of cleavage domains. In this regard, the cleavage domain having double-strand cleavage activity may be used as a cleavage half-domain.

Restriction endonucleases are present in many species and are capable of sequence-specific binding to DNA (at a recognition site), and cleaving DNA at or near the site of binding. Certain restriction enzymes (e.g., Type IIs) cleave DNA at sites removed from the recognition site and have separable binding and cleavage domains. For example, the Type IIs enzyme FokI catalyzes double-stranded cleavage of DNA, at 9 nucleotides from its recognition site on one strand and 13 nucleotides from its recognition site on the other.

Examples of the Type IIs restriction enzymes include FokI, AarI, AceIII, AciI, AloI, BaeI, Bbr7I, CdiI, CjePI, EciI, Esp3I, FinI, MboI, sapl, and SspD51, but are not limited thereto, more specifically, see Roberts et al. (2003) Nucleic acid Res. 31:418-420.

As used herein, the term “fusion protein” refers to a polypeptide formed by the joining of two or more different polypeptides through a peptide bond. The polypeptides contain the zinc finger domain and nucleotide cleavage domain, which can cleave any target site in the nucleotide sequence. Methods for the design and construction of fusion proteins (or polynucleotide encoding fusion protein) may be any methods that are widely known in the art, and the polynucleotide may be inserted into a vector, and the vector may be introduced into a cell. In general, the components of the fusion proteins (e.g., ZFP-FokI fusion) are arranged such that the zinc finger domain is nearest the amino terminus (N-terminus) of the fusion protein, and the cleavage half-domain is nearest the carboxy-terminus (C-terminus). This mirrors the relative orientation of the cleavage domain in naturally-occurring dimerizing cleavage domains such as those derived from the FokI enzyme, in which the DNA-binding domain is nearest the amino terminus and the cleavage half-domain is nearest the carboxy terminus.

As used herein, the term “two pre-determined sites in the genome” means target sites to be cleaved in a genome, and the genome means a set of chromosome having gene clusters. In the present invention, the target sites in the genome are different from each other, and thus each cleavage of different sites could induce deletion, duplication, inversion, replacement or rearrangement to be desired in the present invention.

The “two pre-determined sites in the genome” are the sites cleaved by zinc finger nucleases, and each of the sites is separated by at least 6 base pairs.

In one preferred embodiment, the present invention relates to a method for deleting genomic DNA, comprising the step of cleaving two pre-determined sites in the genome using site-specific nucleases. Preferably, the site-specific nucleases may be zinc finger nucleases, more preferably the zinc finger nuclease may be one or two ZFN pairs, and a ZFN pair may recognize two sites in the genome or each of the two ZFN pairs recognize one of the two different sites in the genome. Far more preferably, two ZFN pairs may be used, and two ZFN pairs may include identical or different zinc finger domains. Most preferably, two ZFN pairs having the different zinc finger domains function to induce targeted deletions of two or more different sites.

Genomic DNA segments that are deleted by ZFNs could be either endogenous chromosomal segments or transgenic cassettes that are inserted into the host genome.

In general, a single zinc finger module is able to recognize 3 base pairs. The combinatorial ZFN pairs used in the present invention consist of two or more zinc finger modules. Therefore, two target sites in the genome should be separated by at least 6 base pairs, which is a length recognized by using two zinc finger modules. If the DNA segment cleaved between two ZFN target sites is less than 5 base pairs in length, the other pair of zinc finger nucleases could not function on the DNA cleaved by one pair of zinc finger nucleases. In this case, at least six base pairs could be recognized, because each monomer constituting a ZFN pair consists of at least two zinc finger modules.

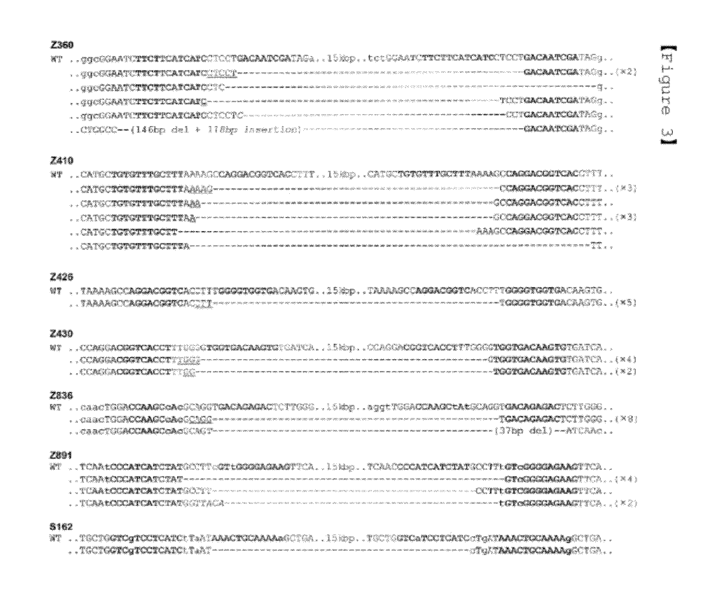

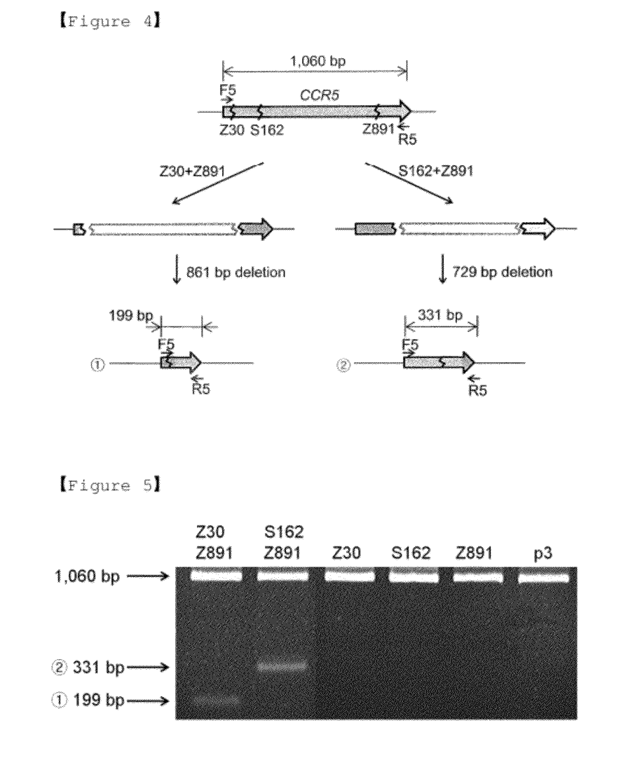

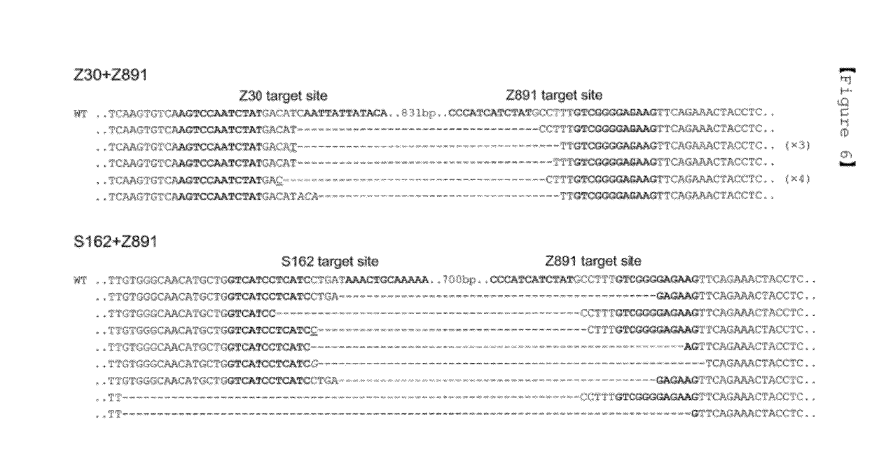

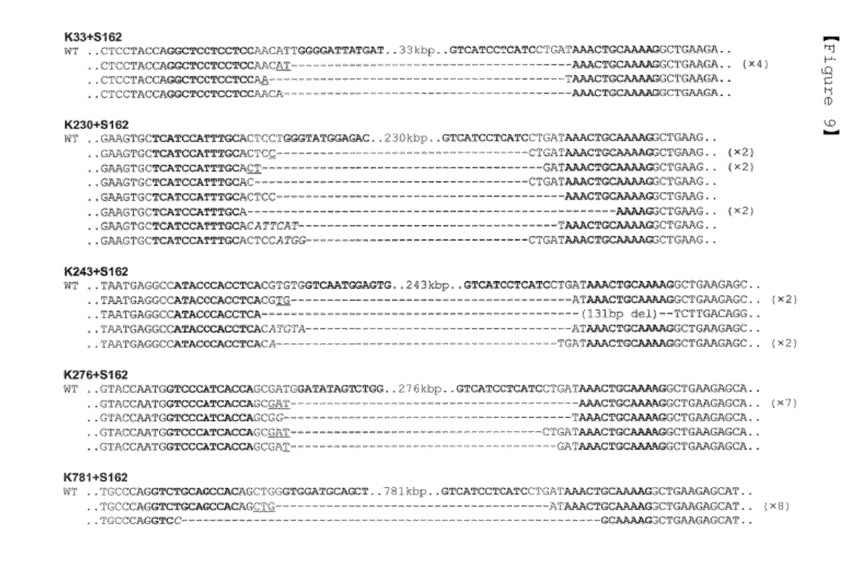

The human chemokine (C-C motif) receptor-encoding CCR5 and CCR2 genes used in the preferred Example 1 of the present invention are located right next to each other on chromosome 3, and are highly homologous. Thus, it was confirmed that ZFNs targeting both the CCR2 and CCR5 sites can induce deletions of 15-kbp DNA segments, indicating that ZFNs targeting two different sites in a chromosome give rise to specific genomic deletions (FIG. 2). As shown in FIG. 3, frequently observed small insertions/deletions at breakpoint junctions suggest that the spacer sequences of the two ZFN target sites do not need to be identical. This indicates that zinc finger domains capable of recognizing two specific sites in the genome are selected to induce genomic deletions in a targeted manner. In Example 2, to confirm whether two different zinc finger nucleases are able to induce genomic deletions, two ZFN pairs each targeting one of two non-homologous sites in the same chromosome were employed to perform an experiment. As a result, it was found that genomic deletion between the two sites occurred (FIGS. 4 to 6). In Example 3, it was confirmed that several ZFNs consisting of four zinc finger modules were used to induce large deletions of genomic segments whose sizes ranged from tens of kilo by to tens of mega by (FIGS. 7 to 10).

In one preferred embodiment, the present invention relates to a method for duplicating genomic DNA, comprising the step of cleaving two pre-determined sites in the genome using site-specific nucleases. Preferably, the site-specific nucleases may be zinc finger nucleases. The characteristics of zinc finger nucleases are the same as described in the above method for deleting genomic DNA.

When two double strand breaks occur in the same chromosome, genomic deletions are generated by intra-molecular joining. In contrast, when two double strand breaks occur separately in two sister chromatids or in two homologous chromosome, intermolecular joining generates deletion in one chromatid and duplication in the other chromatid. In the present invention, the genomic duplication at pre-determined sites in the genome can generate an increase in gene dosage. Since duplications by zinc finger nuclease are always accompanied by deletions, there are no changes in the number of genes duplicated within one cell, but the number of genes can increase to 3 or 4 during fertilization after meiosis. Therefore, the method of duplication of the present invention can be employed for creating useful individuals by increasing the gene dosage of plants or animals.

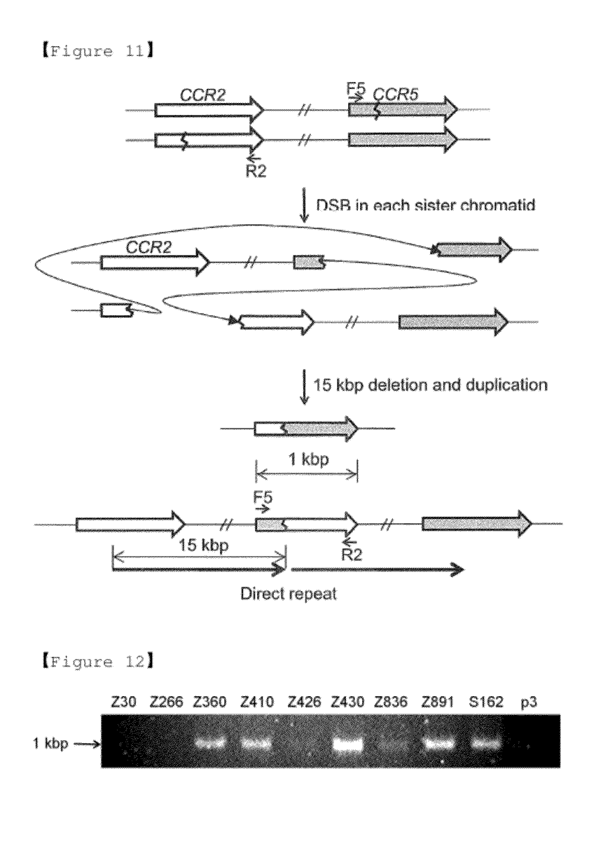

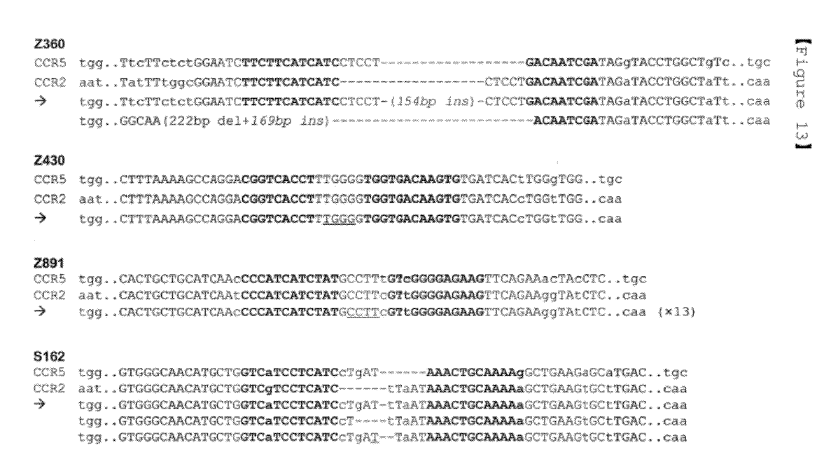

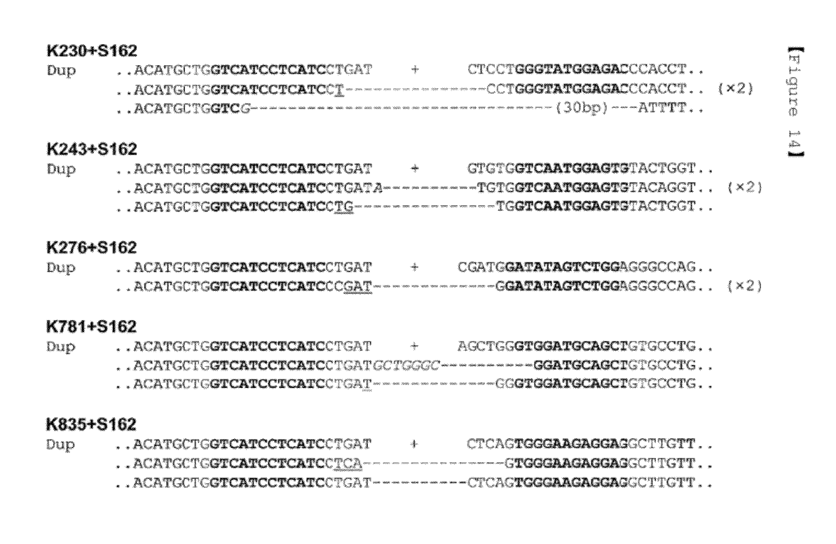

In Example 4, such genomic duplication was confirmed by expressing two ZFN pairs targeting different sites (FIGS. 12 to 14).

In one preferred embodiment, the present invention relates to a method for inverting genomic DNA, comprising the step of cleaving two pre-determined sites in the genome using site-specific nucleases. Preferably, the site-specific nucleases may be zinc finger nucleases. The characteristics of zinc finger nucleases are the same as described in the above method for deleting genomic DNA.

Genomic inversion means that a partial genome is the opposite of what it was before. Genomic inversions are frequently observed in cancer cells and cells of patients with inherited diseases. For example, an inversion of 500 kbp on chromosome 10 was found in thyroid cancer patients exposed to ionizing radiation due to the Chernobyl nuclear accident (Nikiforov et al., 1999; Nikiforova et al., 2000), and the factor VIII inversion on X chromosome was identified in approximately half of all severe hemophilia patients (Lakich et al., 1993). The method of genomic inversion of the present invention can be effectively employed for the prevention and treatment of the above diseases.

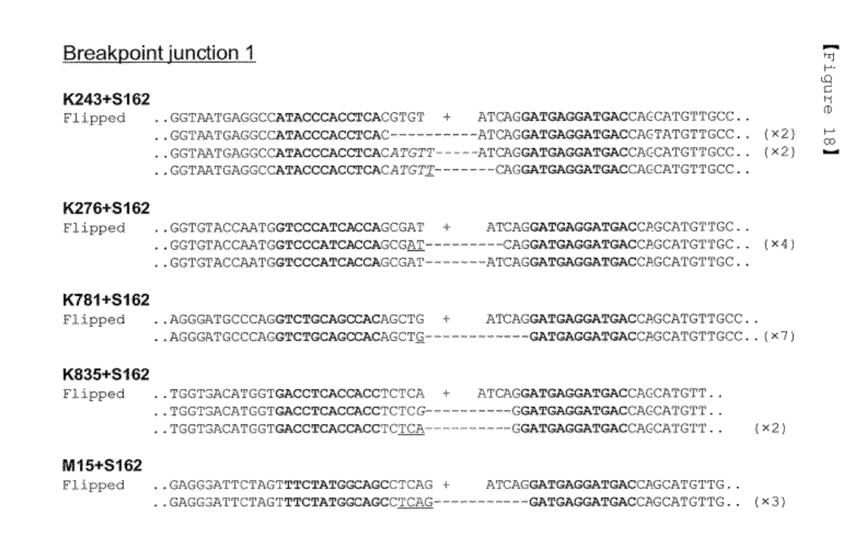

In Example 5, the genomic inversion was confirmed by expressing zinc finger nucleases that target two different sites in the genome using PCR (FIGS. 15 and 16). Unlike I-SceI, the tailor-made ZFNs of the present invention could induce genomic inversion without the prior introduction of target sites in the genome.

In one preferred embodiment, the present invention relates to a method for replacing genomic DNA with synthetic DNA molecules such as double stranded oligonucleotides (dsODNs), comprising the step of cleaving two pre-determined sites in the genome using site-specific nucleases. Preferably, the site-specific nucleases may be zinc finger nucleases. The characteristics of zinc finger nucleases are the same as described in the above method for deleting genomic DNA.

DNA molecules that can be used in the insertion and replacement may include large cloned promoters, enhancers, and genes in addition to synthetic oligonucleotide cassettes. At this time, the 5′ overhangs of DNA to be inserted are made to be complementary to the 5′overhang sequences of the double strand breaks generated by ZFN action, so as to allow foreign DNA insertion in a predetermined direction. The method of genomic replacement of the present invention can be employed to insert strong promoters and enhancers upstream of poorly expressed genes for activation of gene expression or to insert genes of interest downstream of strong promoters for activation of gene expression.

DNA molecules that are used in insertion and replacement can be chemically modified. The 5′ overhangs of dsODNs can be chemically modified to prevent digestion by endogenous exonucleases. For example, phosphothioate modifications of inter-nucleoside linkages can be used to protect overhangs.

The insertion and replacement method can be employed for translocation of a partial genomic DNA. That is, it is possible to replace gene A located on chromosome 1 with gene B located on chromosome 2. To achieve this, two ZFNs targeting each end of gene A and two ZFNs targeting each end of gene B are prepared to be expressed in cells. Such genome shuffling could contribute to study interactions between genes and chromosomes, and be employed for the breed improvement of crops, fish, and livestock animals.

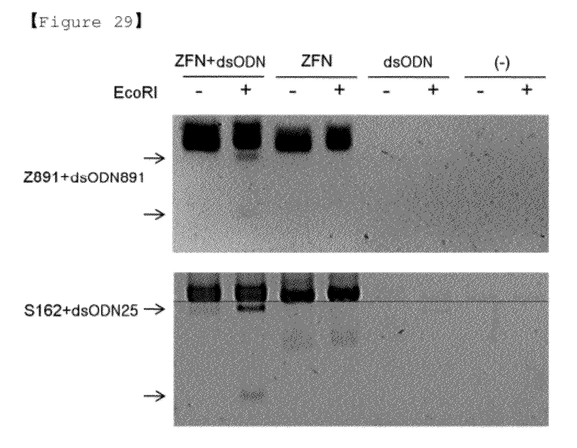

In Example 9, the replacement of a genome was confirmed by investigating whether the replacement can occur as deletion and insertion occur simultaneously in the genome. (FIG. 29)

In another embodiment, the present invention relates to a cell in which genomic

DNA is deleted, duplicated, inverted, replaced, or rearranged, comprising the step of cleaving two pre-determined sites in the genome using site-specific nucleases. Preferably, the site-specific nucleases may be zinc finger nucleases.

The cell may be prokaryotic cells such as E. coli, or eukaryotic cells such as yeast, fungus, protozoa, higher plant, and insect, or amphibian cells, or mammalian cells such as CHO, HeLa, HEK293, and COS-1, for example, cultured cells (in vitro), graft cells and primary cell culture (in vitro and ex vivo), and in vivo cells, and also mammalian cells including human, which are commonly used in the art, without limitation.

In still another embodiment, the present invention relates to a method for inserting synthetic DNA molecules, comprising the step of cleaving a pre-determined site in the genome using site-specific nucleases. Preferably, the site-specific nucleases may be zinc finger nucleases.

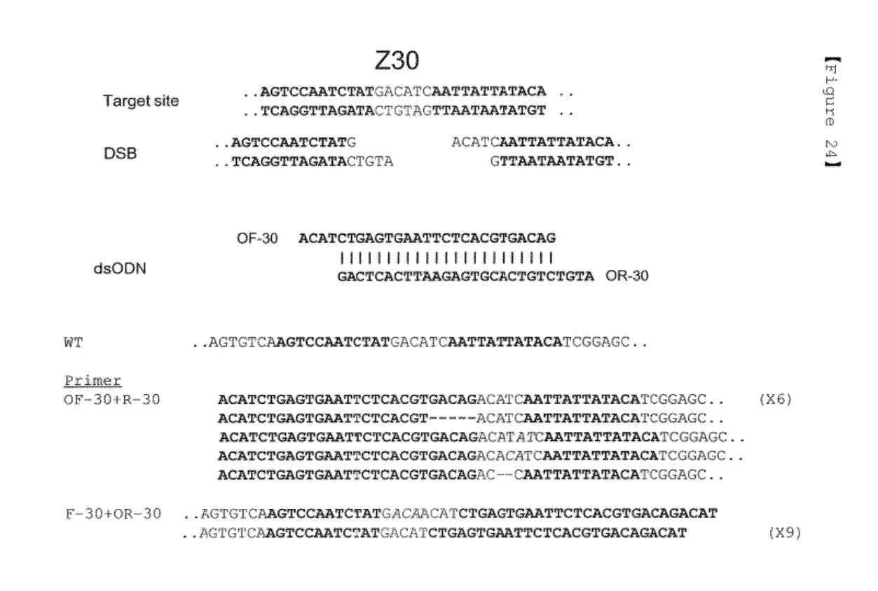

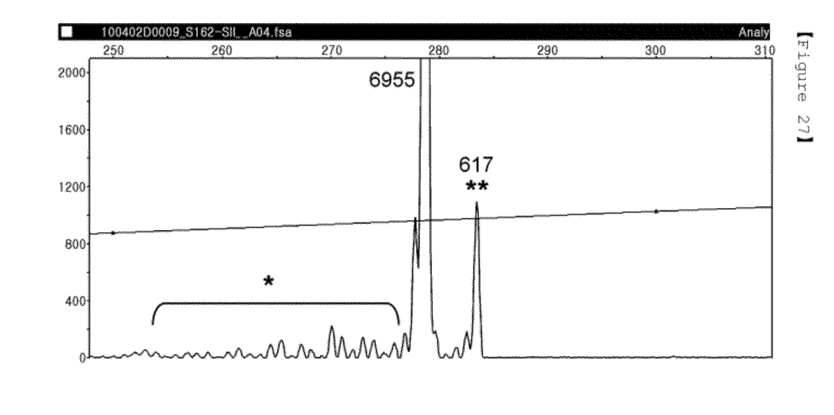

The present inventors inferred the possibility of genomic insertion from genomic DNA deletion in human cultured cells by zinc finger nucleases. In Example 7, synthetic DNA oligonucleotides insertion in a targeted manner was confirmed (FIGS. 23 to 25). Further, whether synthetic DNA oligonucleotides are inserted into the desired sites was confirmed in Example 8 (FIGS. 26 to 28). Unlike the deletion that requires two prior double strand breaks, one prior double strand break is enough for the insertion.

In one preferred embodiment, a synthetic DNA molecule may be prepared by annealing two oligonucleotides that are prepared using a DNA synthesizer. At this time, the synthetic oligonucleotide may be used as it is, or a 5′-phosphate group may be placed on the synthetic oligonucleotide. Preferably, the synthetic DNA molecules prepared by annealing have 5′ 4- or 5-bp overhangs, and the overhang sequences are made to be complementary to the overhang sequences generated by ZFN action.

In one preferred embodiment, a synthetic DNA molecule may be prepared by PCR. Alternatively, the synthetic DNA molecule may be a DNA fragment obtained by restriction enzyme treatment of DNA cloned into a vector such as plasmid.

The length and base sequence of the synthetic DNA molecule can be designed by researchers in an arbitrary manner depending on the desired mutations. For example, a synthetic DNA molecule encoding a stop codon can be inserted within a gene in a genome to interrupt the gene expression, and amino acid sequences such as GFP and FLAG tag can be inserted in frame. The synthetic DNA molecule may be whole or partial cDNA sequences, and may be enhancers, promoters, exons, introns or the like.

The zinc finger nucleases may be a pair of zinc finger nucleases. In one preferred embodiment, the zinc finger nuclease may include two or more zinc finger modules. Preferably, the zinc finger module may be selected from the modules described in Table 1. In the cleavages of nucleotide sequences, the zinc finger nuclease may function as dimers. Preferably, the zinc finger nuclease may be one of those described in Table 3.

The method of inserting or replacing a synthetic DNA in the genome using zinc finger nucleases is advantageous over genome engineering by a gene targeting method via homologous recombination: first, the gene targeting method has a very low efficiency. Second, the gene targeting method requires a gene targeting vector including at least 1 kbp of homology arm at both ends of base sequences to be inserted. Advantageously, the insertion or replacement method using zinc finger nucleases of the present invention does not require gene targeting vectors and also induces mutations with high efficiency.

In still another embodiment, the present invention relates to a cell in which a synthetic DNA insertion occurs, comprising the step of cleaving a pre-determined site in the genome using site-specific nucleases. Preferably, the site-specific nucleases may be zinc finger nucleases.

The synthetic DNA insertion can be generated by the cleavage of one pre-determined site in the genome, unlike deletion, duplication, inversion, or replacement. The use of one ZFN pair is sufficient for insertion. It is possible to insert synthetic DNA at a predetermined site in the genome of higher eukaryotic cells by the insertion method of the present invention with high reproducibility.

In the preferred embodiment, the synthetic DNA insertion was examined by PCR results (FIGS. 23 to 25). In most clones, synthetic DNA cassettes were found to be perfectly inserted into the sites targeted by zinc finger nucleases without any deletions or insertions, indicating that the overhang sequences of synthetic DNA cassettes are made to be complementary to the overhang sequences of the double strand breaks so as to induce site-specific mutations.

In still another embodiment, the present invention relates to site-specific nuclease-induced genome surgery (hereinbelow, referred to as “ZiGS”). Preferably, the site-specific nucleases may be zinc finger nucleases.

The method of the present invention is able to induce targeted deletion and inversion in the genome of the higher eukaryotic cells and individuals using zinc finger nucleases, thereby removing gene clusters from the genome of interest. It often is the case that a single gene knockout does not result in any discernable phenotypic changes in animal studies or in vitro experiments. More often than not, this phenotypic masking is caused by the presence of a homologous gene(s). Interestingly, homologous genes tend to cluster in the genome. For example, CCR2 and CCR5 used in the preferred Example of the present invention are right next to each other on chromosome 3p21. The zinc finger nucleases of the present invention can be used to delete clusters of homologous genes as a unit in the same cell. In addition, the zinc finger nuclease-induced deletion could be used to selectively delete intergenic regions and introns. The zinc finger nucleases could be used to selectively remove disease-associated genes from stem cells or somatic cells. A promoter or an exon could be deleted by targeting two sites in intergenic regions or introns. Furthermore, targeted deletions of DNA segments including the promoter region would completely knock out genes of interest and give rise to a 100% null phenotype. As such, ZiGS can be used to induce genomic deletion or inversion in higher eukaryotic cells and individuals.

In still another embodiment, the present invention relates to a method for expressing the zinc finger nucleases in cells, comprising the steps of (a) determining a specific base sequence to be cleaved; (b) selecting zinc finger modules to recognize the base sequence; (c) preparing zinc finger nucleases including the zinc finger modules of step (b); and (d) introducing the prepared zinc finger nucleases into cells.

The nucleotide sequence of step (a) may exist in or out of cells, and its length is not limited. The nucleotide sequence may exist in circular, single- or double-stranded form.

Step (b) is a step of selecting zinc finger modules to recognize the base sequence, and the zinc finger modules may be any known modules and commercial modules, or may be synthesized newly.

The zinc finger modules of step (c) may include two or more modules, preferably 2 to 4 modules, and more preferably 3 modules. Preferably, the prepared zinc finger nucleases may also be a pair of zinc finger nucleases capable of recognizing different sites, or two pairs of zinc finger nucleases capable of recognizing different sites. More preferably, two pairs of zinc finger nucleases may be prepared.

The method of introducing the zinc finger nucleases into cells of step (d) may be performed by any known method in the art, and a foreign DNA may be introduced into cells by transfection or transduction. The transfection may be performed by a variety of methods known in the art, including calcium phosphate-DNA co-precipitation, DEAE-dextran-mediated transfection, polybrene-mediated transfection, electroporation, microinjection, liposome fusion, lipofection, and protoplast fusion. In step (d), the expression of zinc finger nucleases in cells may be performed by any method known in the art, for example, by using a vector. Examples of the vector may include a plasmid, cosmid, bacteriophage and viral vector, but are not limited thereto. A suitable expression vector may be prepared by including secretory signal sequences as well as regulatory elements such as promoters, operators, initiation codons, termination codons, polyadenylation signals, and enhancers, depending on the purpose.

### REFERENCES

Gomez-Rodriguez, J., Washington, V., Cheng, J., Dutra, A., Pak, E., Liu, P., McVicar, D. W., and Schwartzberg, P. L. (2008). Nucleic Acids Res 36, e117.

Honma, M., Sakuraba, M., Koizumi, T., Takashima, Y., Sakamoto, H., and Hayashi, M. (2007). DNA Repair (Amst) 6, 781-788.

Kim, H. J., Lee, H. J., Kim, H., Cho, S. W., and Kim, J. S. (2009). Genome Res 19, 1279-1288.

Lakich, D., Kazazian, H. H., Jr., Antonarakis, S. E., and Gitschier, J. (1993). Nat Genet 5, 236-241.

Nikiforova, M. N., Stringer, J. R., Blough, R., Medvedovic, M., Fagin, J. A., and Nikiforov, Y. E. (2000). Science 290, 138-141.

Ramirez-Solis, R., Liu, P., and Bradley, A. (1995). Nature 378, 720-724.

Perez, E. E., Wang, J., Miller, J. C., Jouvenot, Y., Kim, K. A., Liu, O., Wang, N., Lee, G., Bartsevich, V. V., Lee, Y. L., et al. (2008). Nat Biotechnol 26, 808-816.

Smith, J., Bibikova, M., Whitby, F. G., Reddy, A. R., Chandrasegaran, S., and Carroll, D. (2000). Nucleic Acids Res 28, 3361-3369.

Valenzuela, D. M., Murphy, A. J., Frendewey, D., Gale, N. W., Economides, A. N., Auerbach, W., Poueymirou, W. T., Adams, N. C., Rojas, J., Yasenchak, J., et al. (2003). Nat Biotechnol 21, 652-659.

All references including patents, patent applications, and others mentioned herein are incorporated by reference in their entirety.

### Mode for the Invention

Hereinafter, the present invention will be described in more detail with reference to Examples. However, these Examples are for illustrative purposes only, and the invention is not intended to be limited by these Examples.

### Example 1

### CCR5-CCR2 Deletion

<1-1> CCR5-CCR2 Deletion

ZFNs targeting the CCR5 gene were produced (Kim et al. 2009). Since many but not all of these CCR5-targeting ZFNs may also show site-specific genome editing activities at the corresponding, homologous sites at the CCR2 locus, it was investigated whether these ZFNs could induce large genomic deletions in addition to site-specific point mutations at each locus.

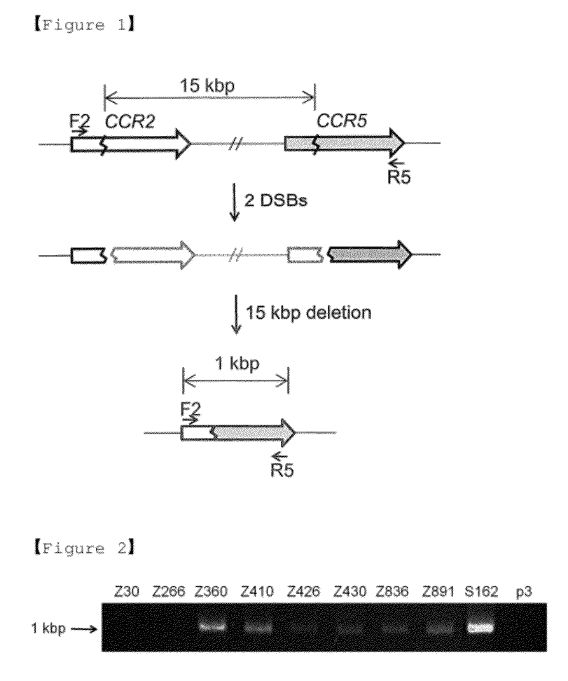

HEK 293 (Human embryonic kidney 293) cells were transfected with ZFN expression plasmids and, after 3 days, genomic DNA was isolated therefrom, and used as a template for DNA amplification to detect genomic deletions. Used were two primers, whose sequences correspond to the CCR2 region or to the CCR5 region and are separated by 16 kbp (FIG. 1). The primer sequences used in the experiment are summarized in the following Table 2.

TABLE 2
Table 2. Primer for PCR

| Primer Name | Sequence (5′ To 3′) | SEQ ID NO. |
|---|---|---|
| F2 | CCACATCTCGTTCTCGGTTT | 18 |
| R2 | GCACCTGCTTTACAGGTTTCT | 19 |
| F5 | ATGGATTATCAAGTGTCAAG | 20 |
| R5 | TCACAAGCCCACAGATATTT | 21 |
| R-S162 | GTATGGAAAATGAGAGCTG | 22 |
| F-K33 | AGCATGGTTCAGAAGGCCAC | 23 |
| R-K33 | TGGCTGAGTAGTATTCCATGGT | 24 |
| F-K230 | GGGAGCTGAAATACCTTCCTT | 25 |
| R-K230 | ATGTGGCATCACACATGGAG | 26 |
| F-K243 | GCCGGGTTTGTACAAGGTAGA | 27 |
| R-K243 | CCCTGTGTTCCCTTCTAAGC | 28 |
| F-K276 | ATCCCTGCCTCACAGCTCAT | 29 |
| R-K276 | TTAGTTCCTGGTTTGGTGCC | 30 |
| F-K781 | TGCAGGTACATGCCGAACTG | 31 |
| R-K781 | CCTACCATCCCCTTTCTCAG | 32 |
| F-K835 | CCCACTGATGCTCTGATAGTTT | 33 |
| R-K835 | TGGGAGATGAAAGGACCTTG | 34 |
| F-M15 | CGAGAAGGAAACCTAGCAAGG | 35 |
| R-M15 | CAATTACTCCCCAGGTGTCC | 36 |

As a result, no PCR product was observed from cells transfected with the control empty plasmid. Amplified DNA segments were observed from cells expressing each of the seven different ZFNs whose target sites are conserved between the CCR5 and CCR2 loci (FIG. 2). The size of the PCR products was about 1 kbp, which was as expected if the DNA segments between the two ZFN target sites were deleted from the chromosome. In contrast, no amplified DNA segments were observed from cells expressing Z30 and Z266 (The numbers indicate the position of the ZFN cleavage sites relative to the initiator codon of the CCR5 gene), whose recognition sites are not conserved at the CCR2 locus, suggesting no genomic deletion occurred in these cells.

These results indicate that ZFNs generating two DSBs (double-strand breaks) in a chromosome but not those generating only one DSB could give rise to large genomic deletions in human cells.

<1-2> Base Sequence Analysis of CCR2-CCR5 Deletion

The PCR products were cloned to determine their DNA sequences, which revealed that, in fact, the CCR2 and CCR5 sites were joined and the intervening 15-kbp DNA segments were deleted (FIG. 3). The base sequences of the breakpoint junctions were consistent with the DNA cleavage patterns of ZFNs. ZFNs function as dimers and each monomer recognizes one of two 9-bp or 12-bp half-sites, which are separated by a 5- or 6-bp spacer. ZFNs cleave DNA at the spacer and generate 5′ 4- or 5-bp overhangs (Smith et al. 2000). The primer sequences used in PCR are summarized in Table 2. Sequence analysis of PCR products showed that a half-site at the CCR2 locus (A) was directly linked to a half-site at the CCR5 locus (B) and that 15-kbp DNA spanning from the other half-site at the CCR2 locus (A) to the other half-site at the CCR5 locus (B) was deleted. The breakpoint junction sequences often showed small (1-14 bp) insertions/deletions in addition to 15-kbp deletions (FIG. 3). These mutagenic patterns of small insertions/deletions are characteristic results from non-homologous end-joining (hereinbelow, referred to as “NHEJ”). In addition, microhomologies of 1-5 bases were observed at the junctions (FIG. 3).

Frequently observed small insertions/deletions at breakpoint junctions suggest that the spacer sequences of the two ZFN target sites do not need to be identical in order to promote genomic deletions. In this regard, it is worth taking note of the CCR5-targeting ZFN of Sangamo (Sangamo Biosciences, Inc.) used in the present invention (this ZFN is designated as ZFN-215 in Perez et al., 2008, but to avoid confusion, is herein denoted as S162). Each of six different ZFNs has the conserved spacer sequences at the CCR5 and CCR2 loci, but S162 generates different overhang sequences at the loci. That is, S162 generates 5′ CTGAT at the CCR5 locus and 5′ ATTAA at the CCR2 locus (their complementary sequences are described in FIG. 3). These overhang sequences are filled or removed during DNA repair. Upon expressing S162, specific 15-kbp genome deletions were observed (FIGS. 2 and 3).

### EXAMPLE 2

### Deletions by Two ZFN Pairs

<2-1> Deletions with Two ZFN Pairs

We next confirmed that two ZFNs targeting two different sites could induce deletions of the intervening DNA segments. For this analysis, two sets of CCR5-targeting ZFNs were chosen, and combinations of Z30+Z891 and S162+Z891 were used for the analysis. Genomic DNA was isolated from cells expressing two ZFNs, and the CCR5 coding sequence was amplified. The Primer sequences used in PCR are summarized in Table 2.

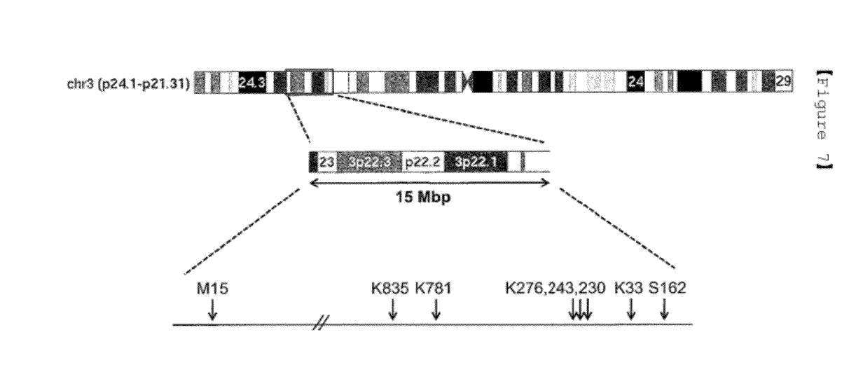

As a result, the amplification of a 1,060-bp DNA corresponding to the entire CCR5 coding region was observed in the wild-type genomic DNA (no DNA deletion), and the amplifications of a 199-bp DNA band and a 331-bp band were observed in cells expressing the Z30+Z891 set and the 5162+Z891 set, respectively (FIGS. 4 and 5). However, the PCR products were not observed in cells expressing no ZFNs or expressing only one ZFN of two ZFNs.

<2-2>Base Sequence Analysis of Deletions by Two ZFN Pairs

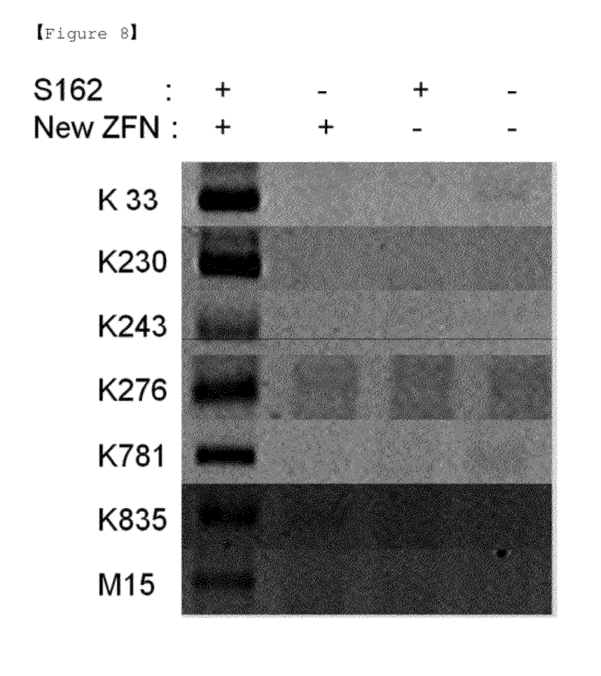

These PCR products were cloned and sequenced, which confirmed specific deletions of approximately 861-bp DNA segments in the cells expressing Z30+Z891 and of approximately 729-bp DNA segments in the cells expressing S162+Z891 (FIG. 6). As noted in Example 1-2, small insertions/deletions and microhomologies in addition to the specific deletions were observed at the joints. These results strongly support the idea that the DNA deletions were mediated via non-homologous end-joining. Even though two ZFNs, each of which targets one of two different sites at the CCR5 locus, were used, targeted deletions were still observed. This ruled out the possibility that homologous recombination is responsible for the deletion events.

As noted in Example 1, the different spacer sequences of two ZFN target sites did not preclude genomic deletions. For example, Z30 and Z891 would generate 5′ ATGT (its complementary sequences are described in FIG. 6) and 5′ CCTT overhangs, respectively, but still gave rise to deletions of approximately 861-bp DNA segments at the CCR5 locus. These results indicate that the design of ZFNs for targeted genome deletions is not limited to the ZFN recognition sites or spacer sequences.

### EXAMPLE 3

### Large Nested Deletions

It was investigated whether it is possible to delete very long stretches of DNA from the human genome using two ZFN pairs. To this end, a series of ZFN pairs was synthesized, whose target sites lie far upstream of the CCR5 locus. 17 naturally-occurring zinc fingers encoded in the human or Drosophila genome were used as modules to assemble 4-finger ZFNs (that is, ZFNs that consist of tandem arrays of four zinc finger modules) (Table 1).

Each of the zinc finger modules recognizes 3-bp sites, and each of the 17 zinc fingers recognizes different 3-bp sub-sites and, collectively, they cover 21 out of 64 triplet sub-sites. Because ZFNs function as dimers, two 4-finger ZFN monomers were prepared per site to prepare ZFN targeting one site. 4-Finger ZFNs recognize two 12-bp half-sites or 24-bp full sites. A total of 30 ZFN pairs were synthesized and these newly prepared ZFNs recognized sites 30 kbp to 46 Mbp upstream of the CCR5 locus.

Each ZFN pair was co-expressed in HEK 293 cells by transfection with the 5162 pair that targets the CCR5 locus. To prevent the formation of swapped dimers, an obligatory heterodimeric form of S 162 was used. Due to alterations of amino acid residues at the dimer interface, the obligatory heterodimer cannot form homodimers (such as AA or BB) or swapped dimers (such as AB) (Miller et al., 2007). Thus, each monomer that constitutes S162 cannot form dimers with any of the new ZFN monomers (unless mentioned otherwise, all of the other ZFNs used in this study were in wild-type form).

As a result, seven of the 30 ZFNs co-expressed with S162 yielded PCR products corresponding to gross deletions (information about these ZFNs is described in the following Table 3). The primer sequences used for PCR are summarized in Table 2.

The ZFNs shown in Table 3 functions as a dimer consisting of two monomers. For example, K33 consists of a pair of K33R and K33F. The name of the zinc finger module described below is derived from four amino acid residues at the main site anticipated as interacting with a base. F1 and F4 refer to the zinc finger module at an N-terminus and one at a C-terminus, respectively. F2 and F3 refer to the zinc finger modules positioned between F1 and F4. F1 binds to 3-bp of 3′-terminus of DNA and F4 to 3-bp of 5′-terminus of DNA. The zinc finger modules are linked to each other by a “TGEKP” amino acid, and F4 and a “Fokl” restriction enzyme domain are linked with(F4H)-TGEK-(FokI QLV).

TABLE 3

| | | | | | Half-site |
|---|---|---|---|---|---|
| ZFN | | | | | sequence |
| name | F1 | F2 | F3 | F4 | (5′ to 3′) |
| K33R | DSCR | QSHR2 | QSHR2 | QSHR2 | GGA GGA GGA GCC |
| K33F | ISNR | VDYK | ISNR | RSHR | GGG GAT TAT GAT |
| K230R | QSHV | QSHR2 | VSNV | QSSR1 | GCA AAT GGA TGA |
| K230F | DSNR | QSHR2 | VDYK | RSHR | GGG TAT GGA GAC |
| K243R | VDYK | RSHR | WSNR | QSHV | TGA GGT GGG TAT |
| K243F | RDER2 | QSHR2 | VSNV | DSAR2 | GTC AAT GGA GTG |
| K276R | DSNR | RDHT | QSHV | RDHT | TGG TGA TGG GTC |
| K276F | RDHT | DSAR2 | QNTQ | ISNR | GAT ATA GTC TGG |
| K781R | DSNR | QSSR1 | VSTR | RDER2 | GTG GCT GCA GAC |
| K781F | VSTR | QSSR1 | ISNR | RDER2 | GTG GAT GCA GCT |
| K835R | DSAR2 | KSNR | WSNR | WSNR | GGT GGT GAG GTC |
| K835F | KSNR | KSNR | QSNR1 | RDHT | TGG GAA GAG GAG |
| M15R | QSNR1 | QNTQ | DSCR | VSTR | GCT GCC ATA GAA |
| M15F | DSNR | RDHT | QSNR1 | QSHR2 | GGA GAA TGG GAC |

Sequence analysis of the PCR products unequivocally corroborated large deletions of 33-kbp, 230-kbp, 276-kbp, 781-kbp, 835-kbp and 15.1-Mbp genomic DNA segments, respectively and small insertions/deletions and microhomologies were also observed (FIGS. 7 to 10). No PCR products were observed in the cells expressing new ZFNs or S162 alone.

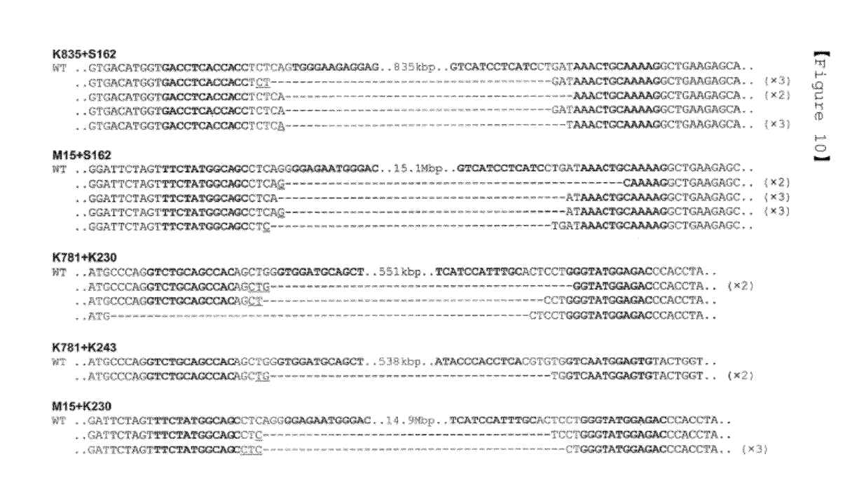

In addition, it was investigated whether the use of new ZFNs in the absence of 5162 could give rise to corresponding genomic deletions. Various combinations of the seven active ZFNs were able to confirm deletions in each case. K230 and M15 gave rise to 14.9 Mbp (=15.1-0.23) deletions. As in Examples 1-2 and 2-2, sequence analysis of the PCR products also revealed that the ZFN-induced deletion is accompanied by microhomologies and small insertions/deletions (FIGS. 9 and 10).

In summary, with various combinations of ZFNs, two (730-bp and 861-bp) deletions within the CCR5 locus, seven different 15-kbp deletions between the CCR2 and CCR5 loci, seven (33-kbp, 230-kbp, 243-kbp, 276-kbp, 781-kbp, 835-kbp and 15.1-Mbp) deletions between the CCR5 locus and loci upstream of CCR5, and three (538-kbp, 551-kbp and 14.9-Mbp) deletions between two loci upstream of CCR5 were observed in human cells. These results suggest that use of any two active ZFNs could give rise to specific genome deletions in human cells.

### EXAMPLE 4

### Duplication

To confirm whether ZFNs induce genomic rearrangements such as duplication or inversion as well as deletion in human cells, the following experiment was performed.

As illustrated in FIG. 11, if double strand breaks occur at the different sites in two sister chromatids, and then joined with each other, genome deletion occurs in one chromatid, and genome duplication occurs in the other chromatid. We used PCR to confirm the occurrence of duplications.

All the ZFNs that target both CCR2 and CCR5 loci gave rise to PCR products corresponding to duplication as shown in FIG. 12. However, amplified DNA segments could not be obtained from cells expressing no ZFNs (negative control cell: represented by p3 in FIG. 12) and cells expressing Z30 and Z266 whose recognition site is present only at the CCR5 locus and not at the CCR2 locus (as shown in FIG. 2, Z30 and Z266 did not induce 15-kbp DNA deletions). The primer sequences used for PCR are summarized in Table 2.

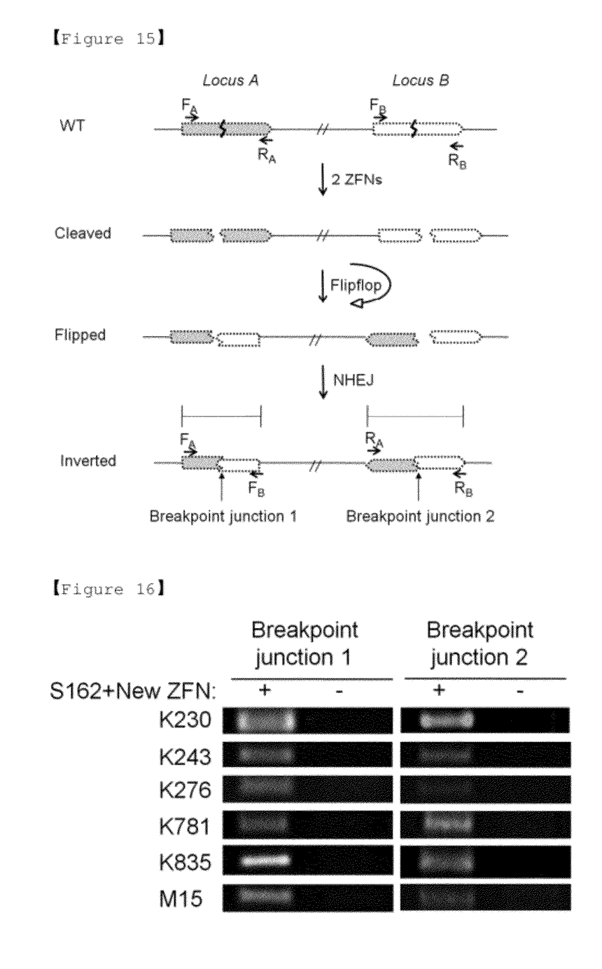

PCR products ware cloned and sequenced, which confirmed the direct connection of 5′ parts of the CCR5 coding region and 3′ parts of the CCR2 coding region (FIG. 13).

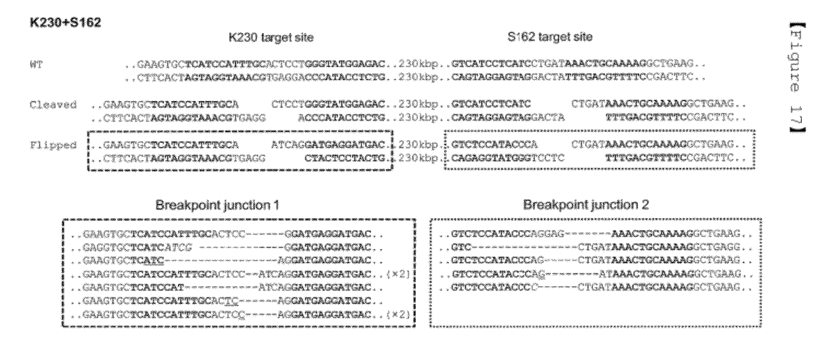

In addition, the experiment was performed with various combinations of ZFNs inducing genome deletion of 30 kbp or more. As a result, PCR products corresponding to the DNA duplication at two ZFN recognition sites were obtained from all of the cells treated with each ZFN combination (data is not shown). Meanwhile, no PCR products were obtained from the cells that were treated with one pair of ZFNs targeting only one site. As shown in deletions, small insertions/deletions and microhomologies were observed at the breakpoint junctions of duplication (FIG. 14). This result suggests that non-homologous end-joining is involved in the ZFN-induced duplications.

### EXAMPLE 5

### Inversion

To investigate the mechanism of inversion and the possibility of reverting any inverted DNA segment in human cells, the present inventors examined the events that occurred after transient expression of ZFNs in HEK 293 cells.

As illustrated in FIG. 15, the ZFN-induced targeted inversion was confirmed by PCR using primers pointing the same direction. The primer sequences used for PCR are summarized in Table 2. If no inversion occurs, no PCR products are obtained, and if inversion occurs, a predetermined size of PCR product can be obtained.

As a result, with various combinations of ZFNs targeting two sites, PCR products corresponding to the genome inversions of 230 kbp, 243 kbp, 276 kbp, 781 kbp, 835 kbp, and 15.1 Mbp in chromosome 3q21 were observed. However, no PCR products were obtained from the cells expressing no ZFNs (FIG. 16).

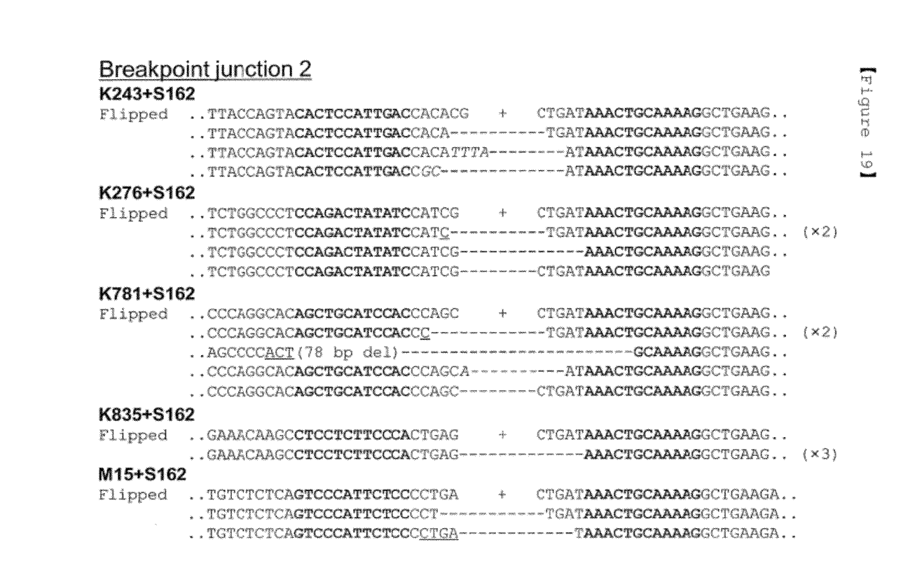

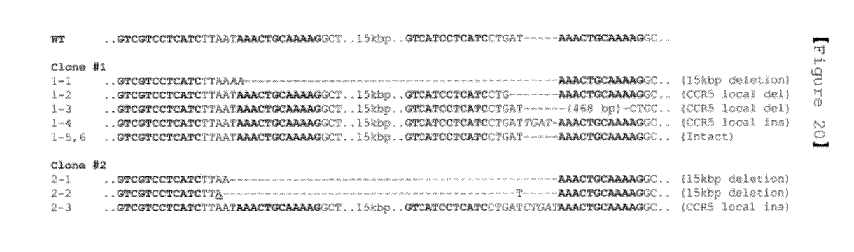

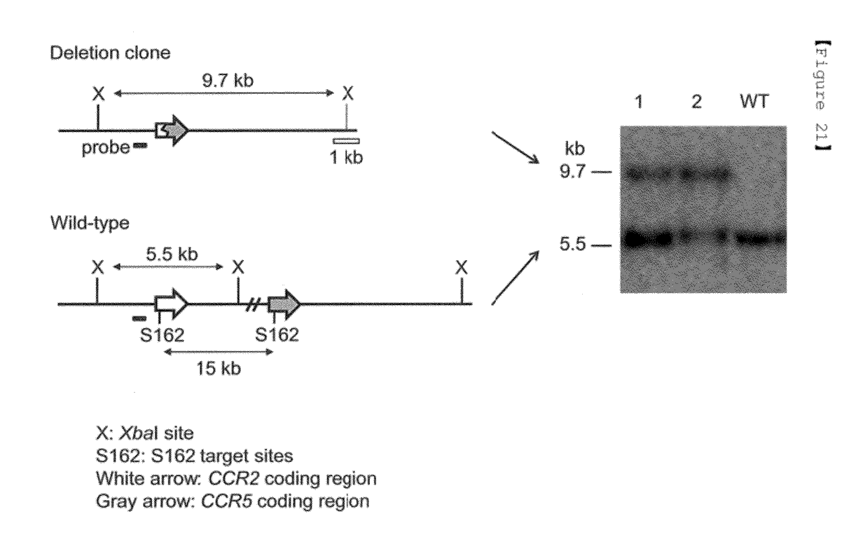

The PCR products were cloned and sequenced, which confirmed that the corresponding regions were indeed inverted (FIGS. 17 to 19). Genome inversions generate two breakpoint junctions. Each of them was sequenced and small insertions/deletions and microhomologies were observed at these junctions. This result suggests that non-homologous end-joining is also involved in the ZFN-induced inversions.

In summary, these results suggest that double strand breaks on chromosome generate genome inversion of the corresponding region, and the inverted genomic segment can be reverted by ZFNs.

### EXAMPLE 6

### Analysis of Clonal Populations of Cells

<6-1> Analysis of Clonal Populations of Cells Using PCR

To investigate the frequencies and characteristics of ZFN-induced genomic deletions, clones in which targeted genome deletions occurs were screened.

S162-treated cells were plated at limiting dilution (0.7 cell/well in a 96-well plate) and cultured for 15 to 21 days. Genomic DNAs were isolated from the separated clonal populations of cells, and were then analyzed by PCR to detect genomic deletions. The primer sequences used for PCR are summarized in Table 2.

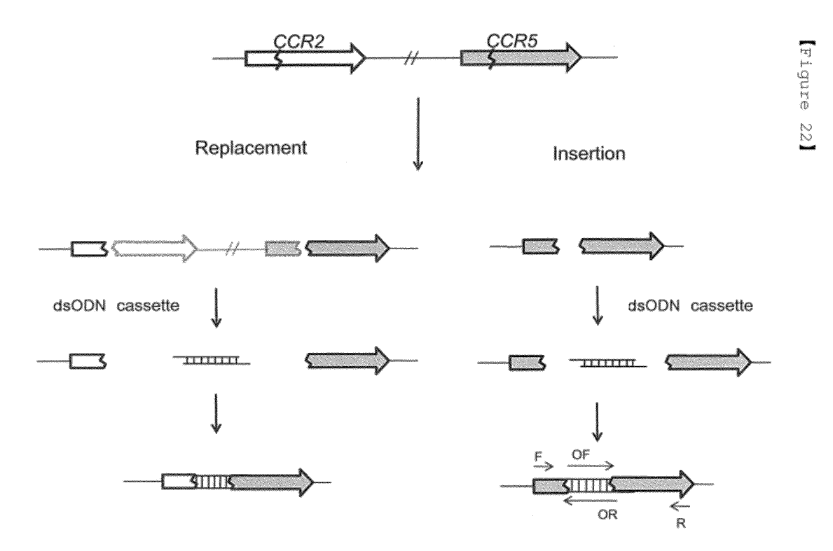

As a result, two clones yielded amplified DNA segments with the expected size. Sequence analysis of these PCR products confirmed specific deletions of 15-kbp DNA segments between two target sites (one site at the CCR2 and the other at CCR5 loci) and joining of two endpoints (FIG. 20). HEK 293 is a multiploid cell line that appears to contain at least three copies of chromosome 3. Clone 2 showed two different base sequences commensurate with 15-kbp deletions, indicating that deletions occurred in two homologous chromosomes. Clone 2 showed no 15-kbp deletions in the other chromosome 3, but showed S 162-induced local mutations at the CCR5 locus. Clone 1 showed a 15-kbp deletion in only one homologous chromosome, and an wild-type base sequence and three different mutations on the other homologous chromosome. We suspect that Clone 1 is not a single clone, but a mixture of two clones. Both Clones 1 and 2 showed no 15-kbp DNA duplications. These results are consistent with the prediction that deletions and duplications will not always occur concurrently.

<6-2>Southern Blot Analysis

These clonal populations of cells were further analyzed by Southern blotting to confirm the presence of genomic deletions. Genomic DNAs were isolated from Clones 1 and 2 and wild-type HEK 293 cells, treated with XbaI and electrophoresed. Genome mutations were examined by using DNAs around the CCR2 locus as a probe.

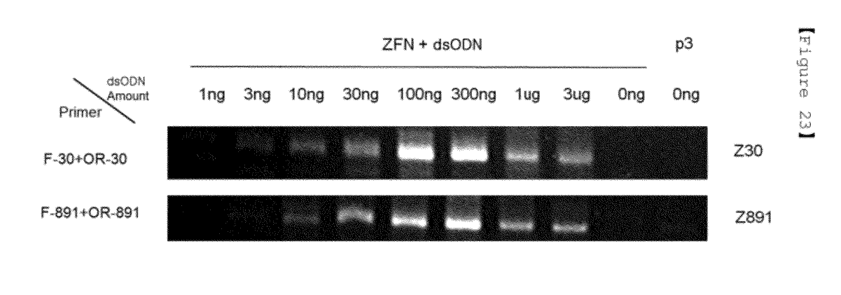

As shown in FIG. 21, DNA bands corresponding to 15-kbp deletions were observed in Clones 1 and 2. Out of dozens of clones analyzed by the present inventors, two clones showed ZFN-induced genome deletions, and efficiency of the ZFN-induced genome deletions was estimated to be 2% or higher. These results indicate that the efficiency is high enough to obtain cells having targeted deletions by ZFN.

### EXAMPLE 7

### Insertion of Synthetic DNA Molecule into Genome

To confirm the possibility of ZFN-induced genome insertion, two strands of small oligonucleotides were synthesized, and annealed to prepare a double stranded ODN (oligodeoxynucleotide) cassette, which was transfected into HEK293 cell together with ZFNs. For this experiment, Z30 and Z891 were used, respectively. Z30 was made to target the CCR5 locus and generate 5′-ACAT and 5′-ATGT overhangs, and the dsODN cassette consists of two complementary 27-mer ODNs which were designed to have 5′ overhangs complementary therewith (represented by OF and OR in FIG. 22). To confirm the insertion of dsODN cassette into Z30-expressing cells, PCR was performed using one of dsODNs constituting the cassette as a primer.

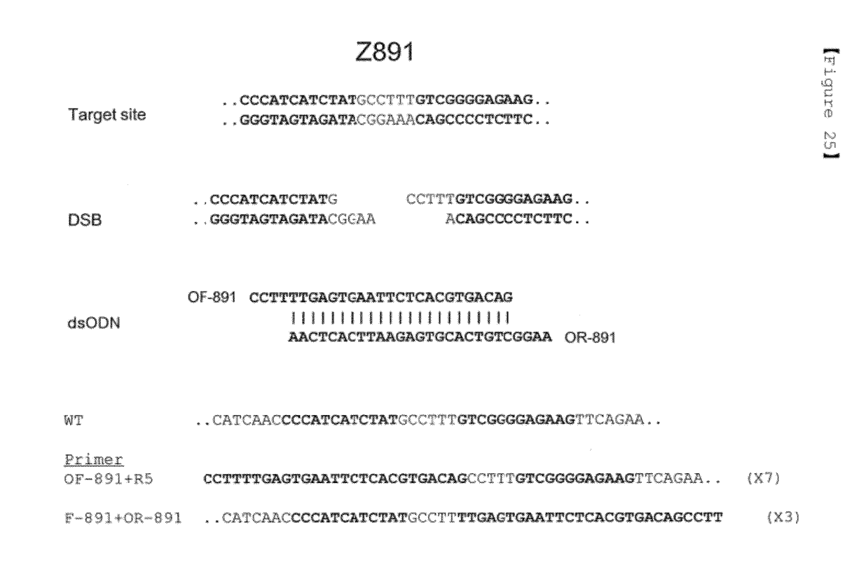

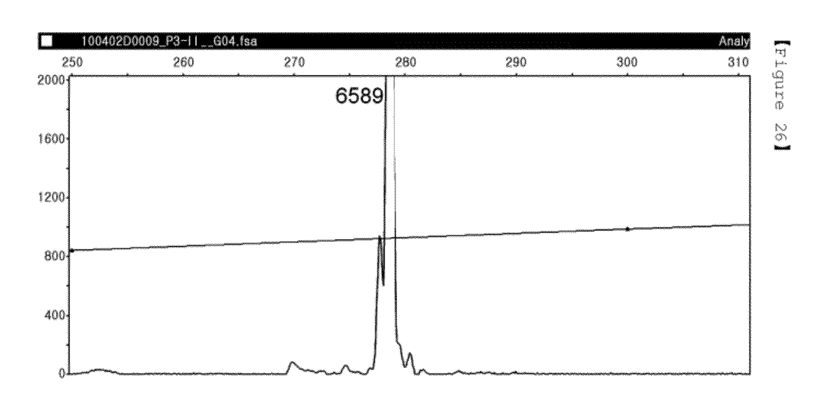

As a result, the expected size of the DNA band was obtained (FIG. 23). The DNA band was cloned and sequenced, which confirmed that the dsODN cassette was virtually inserted into the genome (FIG. 24). Z891 showed the similar results (FIG. 25).

### EXAMPLE 8

### Targeted Insertion of Synthetic DNA Molecules into the Pre-Determined Site in the Genome by ZFN

The present inventors investigated whether it is possible to introduce a double-stranded oligodeoxynucleotide (dsODN) cassette into a predetermined genomic locus in human and any other higher eukaryotic cells and organisms using ZFNs. PCR primers were designed to detect a 280-bp DNA segment at the CCR5 locus, which contains the ZFN (S162) target site. One of the primers was end-labeled with a 6-FAM fluorescent dye. Fluorescent PCR analysis was performed using genomic DNA isolated from cells into which plasmids encoding a ZFN pair (S162) and appropriate double-stranded oligodeoxynucleotides (dsODNs) were co-transfected. Amplified PCR products were analyzed using an ABI 3730x1 DNA analyzer. The resulting data was converted using ABI peak scanner software.

PCR products were obtained using the following primer pair.

Z30F:
(SEQ ID NO. 37)
5′-TGCACAGGGTGGAACAAGATGG-3′
S162R:
(SEQ ID NO. 38)
5′-GAGCCCAGAAGGGGACAGTAAGAAGG-3′

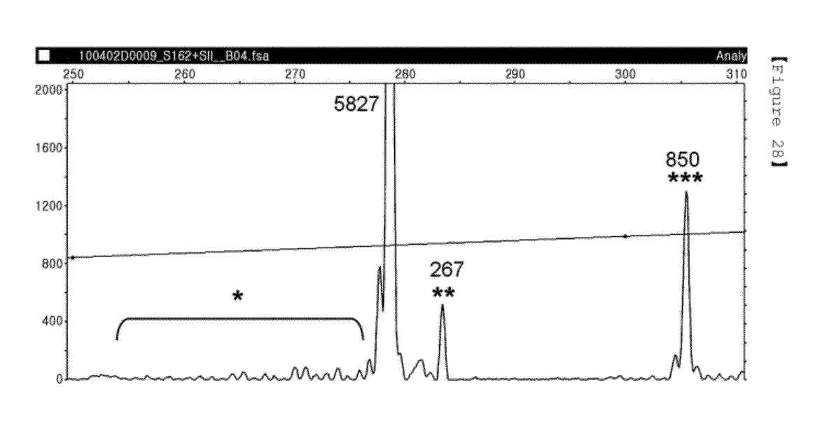

PCR products amplified from genomic DNA isolated from control cells in which ZFN was not expressed showed a single major peak corresponding to the 280-bp DNA segment (FIG. 26). The expression of ZFN induced indels (insertions and deletions) and gave rise to longer and shorter PCR products, which correspond to a 5-bp insertion peak (**) and to deletion peaks (*), respectively (FIG. 27). Of note is the PCR products amplified from cells treated with ZFN and dsODN (FIG. 28). In addition to the 5-bp insertion peak, the fluorescent PCR products showed a new peak (***) that corresponds to the insertion of 28-bp dsODN. These results indicate that ZFNs can be used to insert a synthetic dsODN cassette into a predetermined genomic locus in human cells with high efficiencies.

A dsODN cassette was prepared by annealing the following two ODNs and has 5-bp overhangs at each 5′ end, whose sequences are complementary with the overhangs generated at the genomic ZFN target site.

S162 5F:
(SEQ ID NO. 39)
5′-CTGATTTGAGTGAATTCTCACGTGACAG-3′
S162 5R:
(SEQ ID NO. 40)
5′-ATCAGCTGTCACGTGAGAATTCACTCAA-3′

### EXAMPLE 9

### Replacement

The present inventors investigated whether ZFNs can be used to replace genomic DNA segments with synthetic dsODN cassettes in human cells. The present inventors performed PCR to detect chromosomal DNA segments that resulted from 15-kbp DNA deletions between CCR2 and CCR5 loci using two ZFNs, S162 and Z891, respectively. A dsODN cassette that contained the EcoRI site was co-transfected with the plasmids encoding these ZFNs into HEK293 cells. The primers used in this experiment were same as those used for chromosomal deletions in FIG. 1 (SEQ ID NOs. 18 and 21). PCR products were digested with EcoRI, and then analyzed by agarose gel electrophoresis.

When treating the ZFN and dsODN cassettes together, amplified PCR products were treated with EcoRI and then analyzed by electrophoresis. As a result, DNA bands, which can be anticipated when the cassette is inserted, were observed (see the arrow of FIG. 29), while there was no observation of cleaved DNA bands for the same sample not treated with EcoRI. DNA bands cleaved by EcoRI were not observed in the genomic DNA isolated from cells wherein dsODN cassettes were not added, but ZFNs were only expressed. In the cells wherein ZFNs were not expressed, but dsODN cassettes were added, PCR products corresponding to deletions were not even observed, but only bands with sizes indicating deletions were obtained in amplified PCR products. Only PCR products amplified using genomic DNA isolated from cells treated with both ZFN and dsODN cassette yielded EcoRI-dependent DNA cleavage. These results indicate that it is possible to replace genome DNA segments with synthetic dsODN cassettes using ZFNs.

The dsODN cassette used in this study was prepared by annealing the following two dsODNs and has 4-bp or 5-bp overhangs at each 5′ end, whose sequences are complementary with the overhangs generated at the genomic ZFN target site.

F891 ZFN:
F-891,
(SEQ ID NO. 41)
5′-CCTTTTGAGTGAATTCTCACGTGACAG-3′
OR-891,
(SEQ ID NO. 42)
5′-AAGGCTGTCACGTGAGAATTCACTCAA-3′
S162 ZFN:
2-F-162,
(SEQ ID NO. 43)
5′-TTAATTTGAGTGAATTCTCACGTGACAG-3′
5-OR-162,
(SEQ ID NO. 44)
5′-ATCAGCTGTCACGTGAGAATTCACTCAA-3′

1. A method for deletion, duplication, inversion, replacement, insertion or rearrangement of genomic DNA, comprising the step of cleaving two or more pre-determined sites in a genome using one or more pair of site-specific nucleases.

2. The method according to claim 1, wherein the site-specific nucleases are zinc finger nucleases.

3. The method according to claim 2, wherein the zinc finger nucleases comprise two or more zinc finger modules, and said each zinc finger module binds three nucleotides.

4. The method according to claim 3, wherein the zinc finger module is selected from the modules described in Table 1.

5. (canceled)

6. The method according to claim 2, wherein two pairs of zinc finger nucleases include different zinc finger domains, respectively.

7-9. (canceled)

10. A cell in which genomic DNA is deleted, duplicated, inverted, replaced, inserted or rearranged by the method of claim 1, comprising the step of cleaving two or more pre-determined sites in the genome using one or more pair of site-specific nucleases.

11-16. (canceled)

17. The cell according to claim 10, wherein the site-specific nucleases are zinc finger nucleases.

18. The cell according to claim 10, wherein the zinc finger nucleases comprise two or more zinc finger modules, and said each zinc finger module binds three nucleotides.

19. The cell according to claim 10, wherein the site-specific nucleases are one or more pairs of site-specific nucleases, and wherein the length between a first target site and a second target site, each of which is bound by the site-specific nucleases, is 1 kbp or more.

20. The cell according to claim 19, wherein the two target sites have spacer sequences generated by cleaving of the site-specific nucleases, which sequences are not identical.

21. The cell according to claim 18, wherein the zinc finger module is selected from the modules described in Table 1.

22. The method according to claim 1, wherein the site-specific nucleases are one or more pairs of site-specific nucleases, and wherein the length between a first target site and a second target site, each of which is bound by the site-specific nucleases, is 1 kbp or more.

23. The method according to claim 22, wherein the two target sites have spacer sequences generated by cleaving of the site-specific nucleases, which sequences are not identical.

24. A kit for deletion, duplication, inversion, replacement, insertion or rearrangement of genomic DNA, comprising a first site-specific nuclease binding a first target site and a second site-specific nuclease binding a second target site, and wherein the length between the first target site and the second target site is 1 kbp or more.

25. The kit according to claim 24, wherein the site-specific nucleases are zinc finger nucleases.

26. The kit according to claim 25, wherein the zinc finger nucleases comprise two or more zinc finger modules, and said each zinc finger module binds three nucleotides.

27. The kit according to claim 24, wherein the two target sites have spacer sequences generated by cleaving of the site-specific nucleases, which sequences are not identical.

28. The kit according to claim 26, the zinc finger module is selected from the modules described in Table 1.
